(19) United States
(12) Patent Application Publication
(10) Pub. No.: US 2025/0352337 A1
(43) Pub. Date: Nov. 20, 2025

(54) SEALS FOR DELIVERY APPARATUSES

(71) Applicant: EDWARDS LIFESCIENCES CORPORATION, Irvine, CA (US)

(72) Inventors: Kevin Gantz, Laguna Beach, CA (US); Tri D. Tran, Fountain Valley, CA (US); Kurt Kelly Reed, Costa Mesa, CA (US)

(21) Appl. No.: 19/283,765

(22) Filed: Jul. 29, 2025

Related U.S. Application Data
(63) Continuation of application No. PCT/US2024/013286, filed on Jan. 29, 2024.
(60) Provisional application No. 63/482,210, filed on Jan. 30, 2023.
(60) Provisional application No. 63/582,373, filed on Sep. 13, 2023.

(51) Int. Cl. A61F 2/24; A61M 25/00

(52) U.S. Cl. CPC A61F 2/2427; A61M 25/0043

(57) ABSTRACT

Devices and methods for seals are disclosed. As an example, a delivery apparatus comprises a sleeve shaft comprising a first segment and a second segment, wherein the second segment comprises an inwardly-facing outer surface and an outwardly-facing outer surface, wherein the second segment comprises a lubricious coating; and a seal coupled to the second segment of the sleeve shaft.

### CROSS-REFERENCE TO RELATED APPLICATIONS

This application is a continuation of PCT Patent Application No. PCT/US2024/013286, filed on Jan. 29, 2024, which claims the benefit of U.S. Provisional Patent Application No. 63/582,373, filed Sep. 13, 2023, and which also claims the benefit of U.S. Provisional Patent Application No. 63/482,210, filed Jan. 30, 2023, each of these applications being incorporated by reference herein in its entirety.

### FIELD

The present disclosure relates to seals for delivery apparatuses for prosthetic medical devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery or femoral vein) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

In some examples, a docking device can be implanted first within the native valve and can be configured to receive a prosthetic valve and secure (e.g., anchor) the prosthetic valve in a desired position within the native valve. For example, the docking device can form a more circular and/or stable anchoring site at the native valve annulus in which a prosthetic valve can be expanded and implanted. A transcatheter delivery apparatus can be used to deliver the docking device to the implantation site.

### SUMMARY

Described herein are prosthetic heart valves, docking devices, delivery apparatuses, and methods for implanting prosthetic heart valves. The disclosed prosthetic heart valves, docking devices, delivery apparatuses, and methods can, for example, provide passive hemostatic seals around a shaft having an open channel, such that the open channel is sealed independent of a locking mechanism for the shaft. As such, the devices and methods disclosed herein can, among other things, overcome one or more of the deficiencies of typical prosthetic heart valves, docking devices and associated delivery apparatuses.

A delivery apparatus can comprise a handle and one or more shafts coupled to the handle.

In some examples, a delivery apparatus can comprise a handle, a shaft coupled to the handle, and a passive seal coupled to the shaft, wherein the seal provides homeostasis when the shaft moves relative to the seal.

In some examples, a delivery apparatus can comprise a seal housing; a first shaft extending through the seal housing and comprising an outwardly-facing surface and an inwardly-facing surface, wherein the inwardly-facing surface defines an open channel; a second shaft comprising a first segment and a second segment, wherein the first segment is disposed within the open channel, and wherein the second segment extends out of the open channel and is angled relative to the first segment; and a seal coupled to the first shaft, the seal including a first sealing portion and a second sealing portion, wherein the first sealing portion seals a first gap between the seal housing and the outwardly-facing surface of the first shaft, wherein the second sealing portion seals a second gap between the seal housing and the inwardly-facing surface of the first shaft, wherein the seal provides homeostasis when the first shaft moves relative to the seal.

In some examples, a delivery apparatus can comprise a seal housing; a first shaft extending through the seal housing and comprising an outwardly-facing surface and an inwardly-facing surface, wherein the inwardly-facing surface defines an open channel; a second shaft comprising a first segment and a second segment, wherein the first segment is disposed within the open channel, and wherein the second segment extends out of the open channel and is angled relative to the first segment; and a seal assembly coupled to the first shaft, the seal including a first sealing member and a second sealing member, wherein the first sealing member seals a first gap between the seal housing and the outwardly-facing surface of the first shaft, wherein the second sealing member seals a second gap between the seal housing and the inwardly-facing surface of the first shaft, wherein the seal provides homeostasis when the first shaft moves relative to the seal.

In some examples, a delivery apparatus can comprise a seal housing; a shaft extending through the seal housing, wherein the shaft comprises an outer surface, wherein the outer surface comprises an inwardly-facing portion and an outwardly-facing portion; and a sealing member disposed within the seal housing, wherein the sealing member includes an inner surface defining an opening, wherein the shaft extends through the opening of the sealing member, wherein the sealing member includes an inner projection having an engaging surface, wherein the engaging surface seals against the inwardly-facing portion of the outer surface of the shaft, wherein the sealing member provides hemostasis when the shaft moves relative to the sealing member.

In some examples, a delivery apparatus can comprise a sleeve shaft comprising a first segment and a second segment, wherein the second segment comprises an inwardly-facing outer surface and an outwardly-facing outer surface, wherein the second segment comprises a lubricious coating; and a seal coupled to the second segment of the sleeve shaft.

In some examples, a delivery apparatus can comprise a seal housing defining a lubricant chamber including a lubricant; a seal disposed within the seal housing; and a sleeve shaft extending through the seal housing, the sleeve shaft comprising a first segment and a second segment, wherein the second segment extends through the lubricant chamber and the seal, wherein the second segment comprises an inwardly-facing outer surface and an outwardly-facing outer surface.

A seal assembly for a delivery apparatus can comprise multiple sealing members that are coupled together and form a seal therebetween, wherein each sealing member defines an axially-extending opening.

In some examples, a seal assembly for a delivery apparatus can comprise a first sealing member, the first sealing member defining a first opening extending through the first sealing member in an axial direction, wherein the first opening comprises an inwardly-facing surface, the inwardly-facing surface configured to seal against an outwardly-facing surface of a shaft; and a second sealing member coupled to the first sealing member, the second sealing member defining a second opening extending through the second sealing member in the axial direction, wherein the second sealing member comprises an inner projection extending into the second opening in a radial direction, wherein the inner projection includes an outwardly-facing, engaging surface, wherein the engaging surface is configured to seal against an inwardly-facing surface of the shaft.

A seal for a delivery apparatus can comprise a body defining an opening for a shaft, wherein the body includes an inwardly-facing surface and an outwardly-facing surface configured to seal against the shaft.

In some examples, a seal for a delivery apparatus can comprise a body, the body having a first sealing portion and a second sealing portion, the first sealing portion axially spaced apart from the second sealing portion, wherein the first sealing portion comprises an opening having inwardly-facing surface, and wherein the second sealing portion comprises an inner projection having an outwardly-facing surface.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a stage in an example mitral valve replacement procedure where a guide catheter and a guidewire are inserted into a blood vessel of a patient and navigated through the blood vessel and into a heart of the patient, towards a native mitral valve of the heart.

FIG. 2A schematically illustrates another stage in the example mitral valve replacement procedure where a docking device delivery apparatus extending through the guide catheter is implanting a docking device for a prosthetic heart valve at the native mitral valve.

FIG. 2B schematically illustrates another stage in the example mitral valve replacement procedure where the docking device of FIG. 2A is fully implanted at the native mitral valve of the patient and the docking device delivery apparatus has been removed from the patient.

FIG. 3A schematically illustrates another stage in the example mitral valve replacement procedure where a prosthetic heart valve delivery apparatus extending through the guide catheter is implanting a prosthetic heart valve in the implanted docking device at the native mitral valve.

FIG. 3B schematically illustrates another stage in the example mitral valve replacement procedure where the prosthetic heart valve is fully implanted within the docking device at the native mitral valve and the prosthetic heart valve delivery apparatus has been removed from the patient.

FIG. 4 schematically illustrates another stage in the example mitral valve replacement procedure where the guide catheter and the guidewire have been removed from the patient.

FIG. 5 is a side view of a docking device, according to one example.

FIG. 6A is side view of a delivery apparatus for a docking device, according to one example.

FIG. 6B depicts a portion of the delivery apparatus of FIG. 6A.

FIG. 7 depicts a portion of a shaft assembly of the delivery apparatus of FIG. 6A.

FIG. 8 is a perspective view of an active hemostatic seal configured to seal around a sleeve shaft of a delivery apparatus for a docking device.

FIG. 9 is a perspective view of the active hemostatic seal of FIG. 8 positioned within a seal housing around the sleeve shaft.

FIG. 10 is a cross-sectional side view of a passive hemostatic seal assembly positioned within a seal housing, according to an example.

FIG. 11 is a perspective view of the seal assembly of FIG. 10.

FIGS. 12A-13B are additional views of sealing members of the seal assembly of FIG. 10.

FIG. 14 is a perspective view of the seal assembly of FIG. 10 coupled to the sleeve shaft.

FIG. 15 is a perspective view of a segment of the seal housing of FIG. 10.

FIG. 16 is a cross-sectional side view of a passive hemostatic seal positioned within a seal housing, according to an example.

FIG. 17 is a perspective view of the seal of FIG. 16 with a cap of the seal housing removed for illustration purposes.

FIGS. 18A-18D are additional views of a sealing member of the seal of FIG. 16.

FIG. 19 is a perspective view of the seal of FIG. 16 coupled to a sleeve shaft.

FIG. 20 is a cross-sectional perspective view of the seal of FIG. 16 coupled to a sleeve shaft.

FIG. 21 is a perspective view of a passive hemostatic seal, according to one example.

FIG. 22 is an end view of the seal of FIG. 21.

FIG. 23 is a side view of a proximal portion of a sleeve shaft, according to an example.

FIG. 24 is a cross-sectional view of the sleeve shaft, taken along section 24-24 of FIG. 23.

FIG. 25 is cross-sectional side view of a passive hemostatic seal assembly and lubricant chamber positioned within a seal housing, according to an example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Introduction to the Disclosed Technology

Described herein are examples of a steerable delivery apparatus (sometimes referred to as a steerable catheter) that can be used to navigate a subject's vasculature to deliver an implantable medical device (e.g., a prosthetic heart valve, a docking device), tools, agents, or other therapy to a location within the body of a subject. Examples of procedures in which the steerable catheters are useful include neurological, urological, gynecological, fertility (e.g., in vitro fertilization, artificial insemination), laparoscopic, arthroscopic, transesophageal, transvaginal, transvesical, transrectal, and procedures including access in any body duct or cavity. Particular examples include placing implants, including stents, grafts, embolic coils, and the like; positioning imaging devices and/or components thereof, including ultrasound transducers; and positioning energy sources, for example, for performing lithotripsy, RF sources, ultrasound emitters, electromagnetic sources, laser sources, thermal sources, and the like.

In connection therewith, various systems, apparatuses, methods, or the like are described herein that, in some examples, can create a passive seal for a shaft of a delivery apparatus, such that the shaft is sealed (e.g., homeostatic seal) as the shaft translates relative to other components of the delivery apparatus.

### Examples of the Disclosed Technology

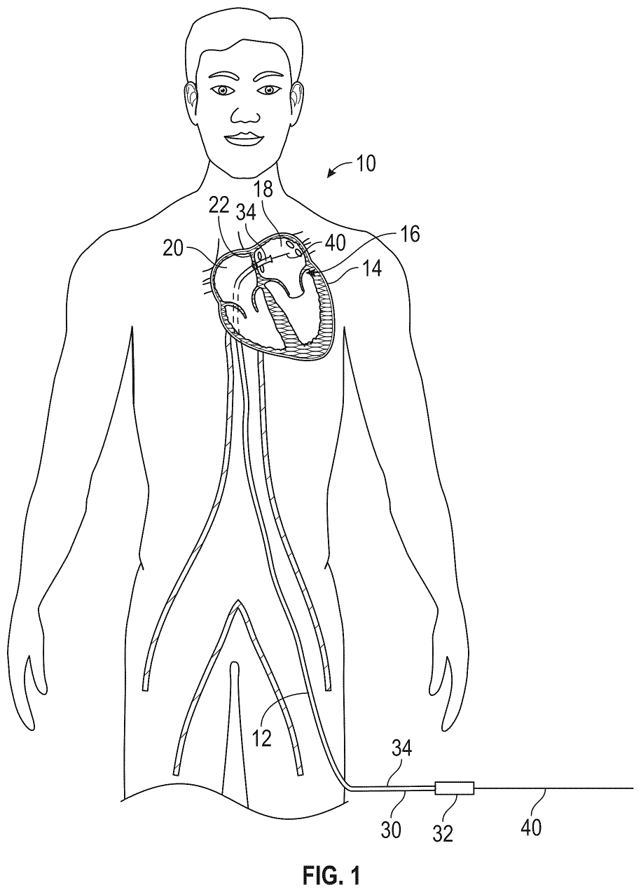

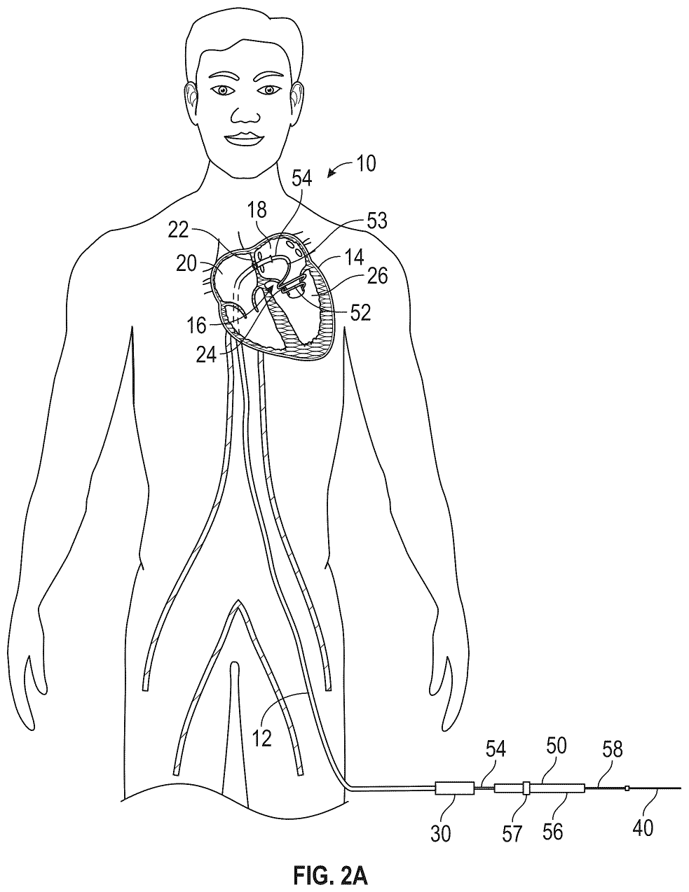

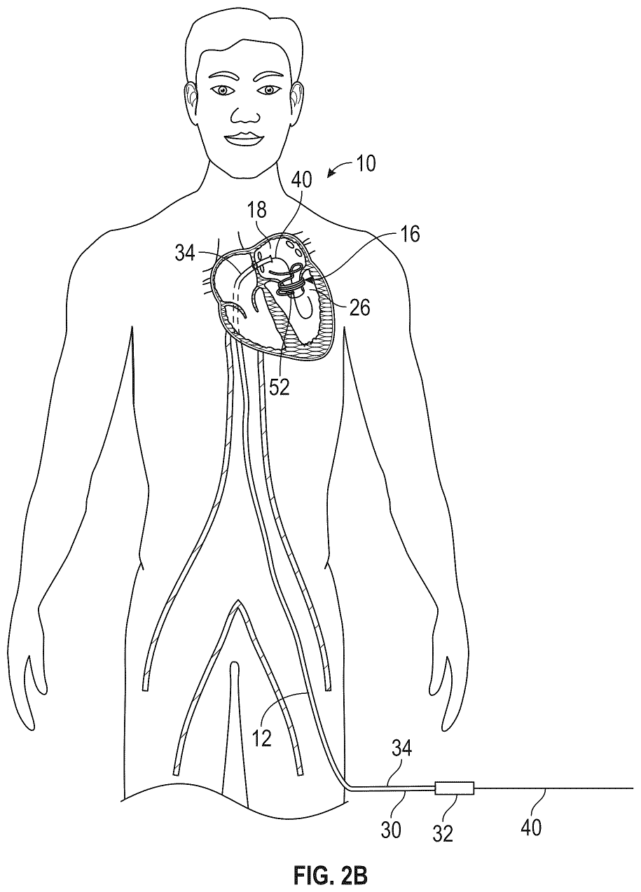

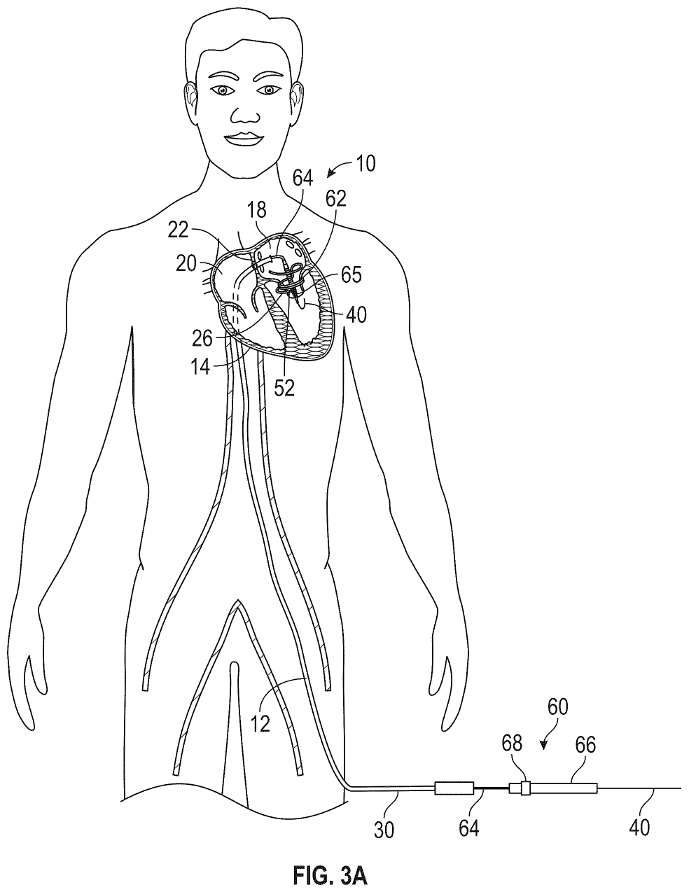

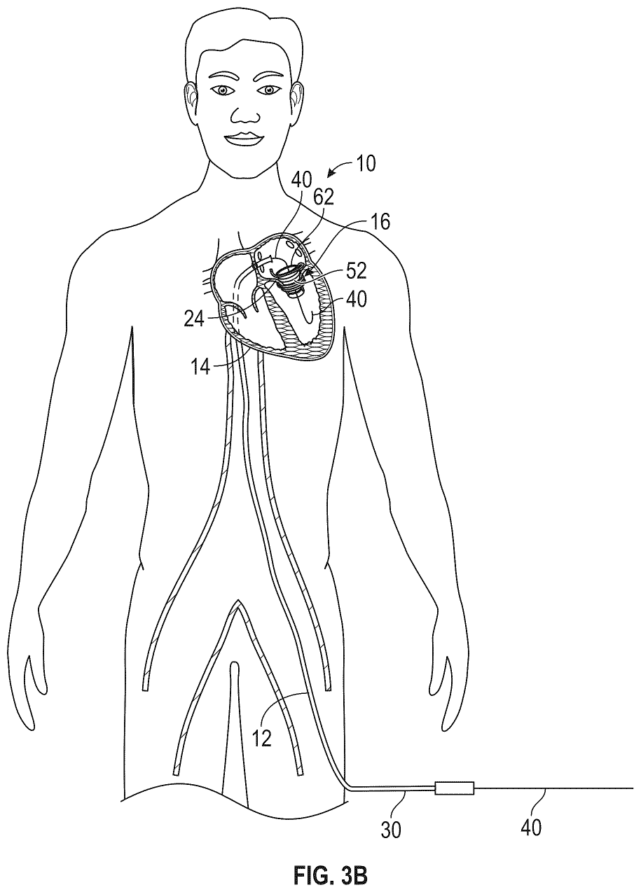

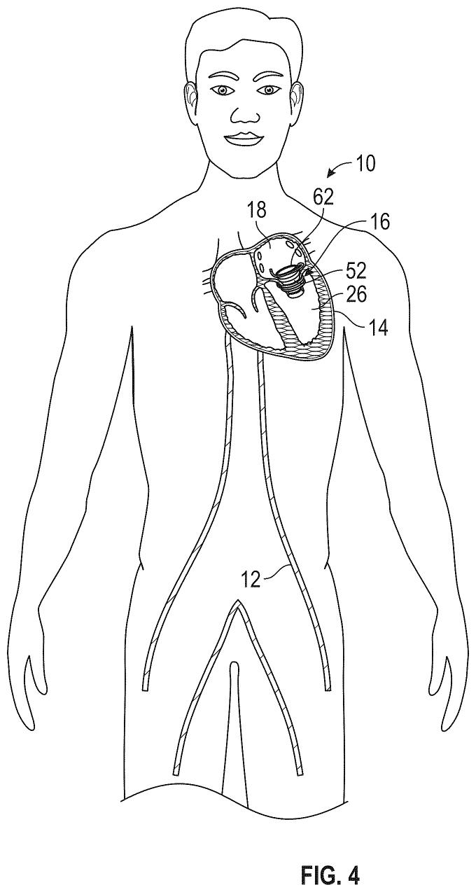

FIGS. 1-4 depict an example of a transcatheter heart valve replacement procedure (e.g., a mitral valve replacement procedure) which utilizes a docking device 52 and a prosthetic heart valve 62, according to one example. During the procedure, a user first creates a pathway to a patient's native heart valve using a guide catheter 30 (FIG. 1). The user then delivers and implants the docking device 52 at the patient's native heart valve using a docking device delivery apparatus 50 (FIG. 2A) and then removes the docking device delivery apparatus 50 from the patient 10 after implanting the docking device 52 (FIG. 2B). The user then implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (the native mitral valve 16 or native mitral valve annulus). As shown, the heart 14 is illustrated schematically. For example, the anterior leaflet and chordae of the native mitral valve 16 are omitted for illustration purposes, such that only a portion of the posterior leaflet of the native mitral valve 16 is illustrated.

Initially, the user may first make an incision in the patient's body to access the blood vessel 12. For example, in the example illustrated in FIG. 1, the user may make an incision in the patient's groin to access a femoral vein. Thus, in such examples, the blood vessel 12 may be a femoral vein.

After making the incision at the blood vessel 12, the user may insert the guide catheter 30, the guidewire 40, and/or additional devices (such as an introducer device or transseptal puncture device) through the incision and into the blood vessel 12. The guide catheter 30 (which can also be referred to as an “introducer device,” “introducer,” or “guide sheath”) is configured to facilitate the percutaneous introduction of various implant delivery devices (e.g., the docking device delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (e.g., the guide catheter 30, the docking device delivery apparatus 50, the prosthetic valve delivery apparatus 60, additional catheters, or the like) and their associated devices (e.g., docking device, prosthetic heart valve, and the like) to the implantation site within the heart 14, and thus may extend all the way through the blood vessel 12 and into a left atrium 18 of the heart 14 (FIG. 1) and in some examples, through the native mitral valve 16 and into a left ventricle of the heart 14.

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (e.g., through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts another stage in the example mitral valve replacement procedure where a docking device 52 is being implanted at the native mitral valve 16 of the heart 14 of the patient 10 using a docking device delivery apparatus 50 (which may also be referred to as an “implant catheter” and/or a “docking device delivery device”).

In general, the docking device delivery apparatus 50 comprises a delivery shaft 54, a handle 56, and a pusher assembly 58. The delivery shaft 54 is configured to be advanced through the patient's vasculature (blood vessel 12) and to the implantation site (e.g., native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the docking device delivery apparatus 50 is configured to be gripped and/or otherwise held by the user, outside the body of the patient 10, to advance the delivery shaft 54 through the patient's vasculature (e.g., blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (e.g., the native mitral valve 16). For example, the pusher assembly 58 is configured to be adjusted by the user to push the docking device 52 out of the distal end portion 53 of the delivery shaft 54. A shaft of the pusher assembly 58 can extend through the delivery shaft 54 and can be disposed adjacent to the docking device 52 within the delivery shaft 54. In some examples, the docking device 52 can be releasably coupled to the shaft of the pusher assembly 58 via a connection mechanism of the docking device delivery apparatus 50 such that the docking device 52 can be released after being deployed at the native mitral valve 16.

Further details of the docking device delivery apparatus and its variants are described in International Publication No. WO2020/247907, which is incorporated by reference herein in its entirety.

Referring again to FIG. 2A, after the guide catheter 30 is positioned within the left atrium 18, the user may insert the docking device delivery apparatus 50 (e.g., the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the docking device delivery apparatus 50 through the blood vessel 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (e.g., pushing) the handle 56 of the docking device delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (e.g., the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (e.g., the portion of the docking device 52 shown in FIG. 2A that is configured to be positioned within a left ventricle 26 and/or on the ventricular side of the native mitral valve 16), the user may then deploy the remaining portion of the docking device 52 (e.g., an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the docking device delivery apparatus 50 from the docking device 52. Once the docking device 52 is disconnected from the docking device delivery apparatus 50, the user may retract the docking device delivery apparatus 50 out of the blood vessel 12 and away from the patient 10 so that the user can deliver and implant a prosthetic heart valve 62 within the implanted docking device 52 at the native mitral valve 16.

FIG. 2B depicts this stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts another stage in the mitral valve replacement procedure where the user is delivering and/or implanting a prosthetic heart valve 62 (which can also be referred to herein as a “transcatheter heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient's vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient's vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (e.g., the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (e.g., pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (e.g., inflate the inflatable balloon), thereby radially expanding the prosthetic heart valve 62 within the docking device 52.

FIG. 3B shows another stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts another stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (e.g., tricuspid, pulmonary, and/or aortic valves). Further, the same and/or similar delivery apparatuses (e.g., docking device delivery apparatus 50, prosthetic valve delivery apparatus 60, guide catheter 30, and/or guidewire 40), docking devices (e.g., docking device 52), replacement heart valves (e.g., prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the docking device delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned/disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

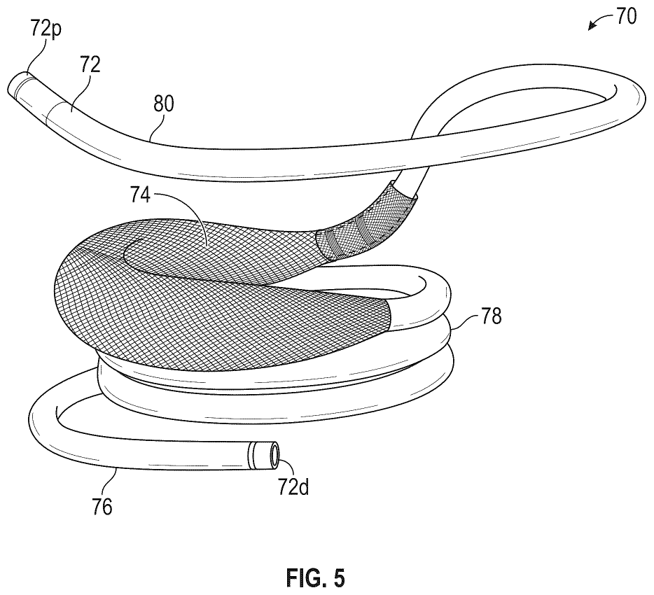

FIG. 5 illustrates the docking device 52 in greater detail. As depicted in FIG. 5, the docking device 52 in its deployed configuration can be configured to receive and secure a prosthetic valve within the docking device, thereby securing the prosthetic valve at the native valve annulus.

The docking device 52 can comprise a coil 72 and an optional guard member 74 covering at least a portion of the coil 72. In certain examples, the coil 72 can include a shape memory material (e.g., nickel titanium alloy or “Nitinol”) such that the docking device 52 (and the coil 72) can move from a substantially straight configuration (or delivery configuration) when disposed within the delivery shaft 54 of the delivery apparatus 50 to a helical, deployed configuration after being removed from the delivery shaft 54.

The coil 72 has a proximal end 72p and a distal end 72d (which also respectively define the proximal and distal ends of the docking device 52). When being disposed within the delivery shaft 54 (e.g., during delivery of the docking device 52 into the vasculature of a patient), a body of the coil 72 between the proximal end 72p and distal end 72d can form a generally straight delivery configuration (i.e., without any coiled or looped portions, but can be flexed or bent) so as to maintain a small radial profile when moving through a patient's vasculature. After being removed from the delivery shaft 54 and deployed at an implant position, the coil 72 can move from the delivery configuration to the helical deployed configuration and wrap around native tissue adjacent the implant position. For example, when implanting the docking device at the location of a native valve, the coil 72 can be configured to surround native leaflets of the native valve (and the chordae tendineae that connects native leaflets to adjacent papillary muscles, if present).

The docking device 52 can be releasably coupled to the docking device delivery apparatus 50. For example, in certain examples, the docking device 52 can be coupled to a delivery apparatus (as described above) via a release suture that can be configured to be tied to the docking device 52 and cut for removal.

As shown in FIG. 5, the coil 72 in the deployed configuration can include a leading turn 76 (or “leading coil”), a central region 78, and a stabilization turn 80 (or “stabilization coil”) around a central longitudinal axis. The central region 78 can possess one or more helical turns having substantially equal inner diameters. The leading turn 76 can extend from a distal end of the central region 78 and has a diameter greater than the diameter of the central region 78, in the illustrated example. The stabilization turn 80 can extend from a proximal end of the central region 78 and has a diameter greater than the diameter of the central region 78, in the illustrated example.

Further details of the docking device and its variants are described in International Application No. PCT/US2021/056150, which is incorporated by reference herein in its entirety.

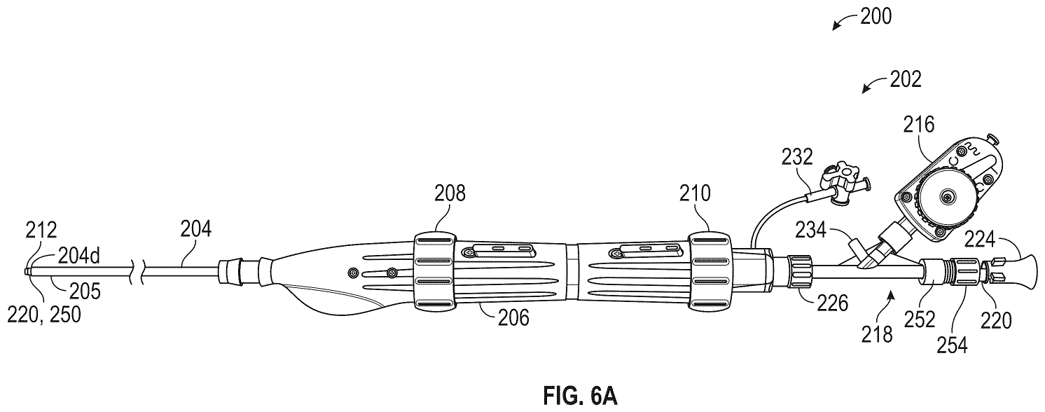

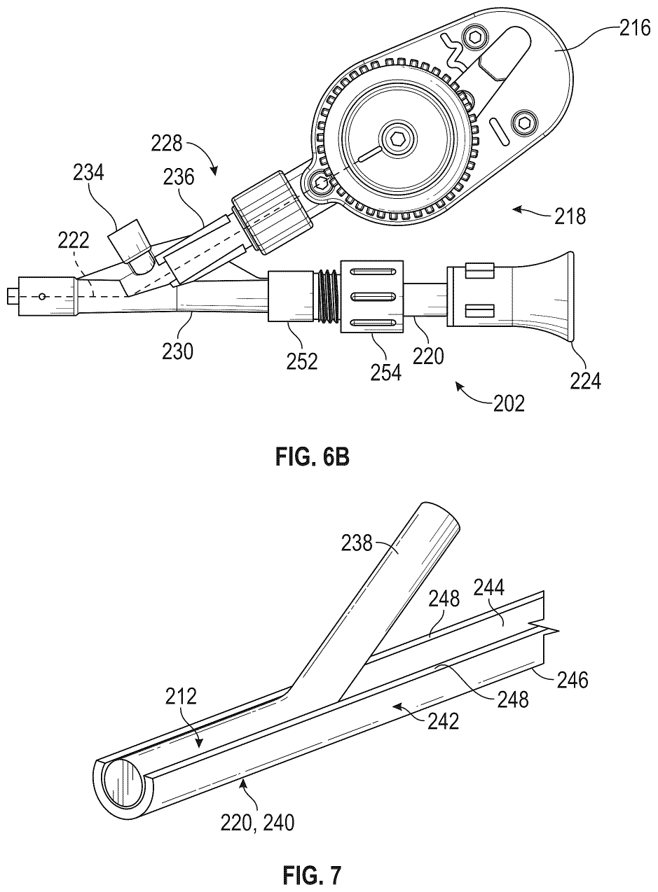

FIG. 6A illustrates a delivery apparatus 200 configured to implant a docking device, such as docking device 52 (FIG. 5) or other docking devices, to a target implantation site in a patient, according to one example. For example, the delivery apparatus 200 can be used as the docking device delivery apparatus 50 in a prosthetic valve implantation procedure, as described above with reference to FIG. 2A. The delivery apparatus 200 can also be referred to as a “docking device delivery apparatus,” “dock delivery catheter,” or “dock delivery system.”

As shown, the delivery apparatus 200 can include a handle assembly 202 and a delivery shaft 204 (also referred to as the “delivery sheath” or “outer shaft” or “outer sheath”) extending distally from the handle assembly 202. The handle assembly 202 can include a first or main handle 206 including one or more knobs, buttons, wheels, and/or other means for controlling and/or actuating one or more components of the delivery apparatus 200. For example, in some examples, as shown in FIG. 6A, the main handle 206 can include knobs 208 and 210 which can be configured to steer or control flexing of the delivery apparatus 200 such as the delivery shaft 204 and/or the sleeve shaft 220 described below.

In certain examples, the delivery apparatus 200 can also include a pusher shaft 212 and a sleeve shaft 220, both of which can extend through an inner lumen of the delivery shaft 204 and have respective proximal end portions extending into the handle assembly 202.

As described below, a distal end portion (also referred to as “distal section”) of the sleeve shaft 220 can be configured to cover (e.g., surround) the docking device 52 (see FIG. 5). For example, the distal end portion of the sleeve shaft 220 can comprise a generally tubular structure. The docking device 52 can be retained inside the sleeve shaft 220, which is further retained by a distal end portion 205 of the delivery shaft 204, when navigating through a patient's vasculature.

Additionally, the distal end portion 205 of the delivery shaft 204 can be configured to be steerable. In one example, by rotating a knob (e.g., 208 or 210) on the main handle 206, a curvature of the distal end portion 205 can be adjusted so that the distal end portion 205 of the delivery shaft 204 can be oriented in a desired angle. For example, to implant the docking device 52 at the native mitral valve location, the distal end portion 205 of the delivery shaft 204 can be steered in the left atrium so that at least a portion of the sleeve shaft 220 and the docking device 52 retained therein can extend through the native mitral valve annulus at a location adjacent the posteromedial commissure.

In certain examples, the pusher shaft 212 and the sleeve shaft 220 can be coaxial with one another, at least within the delivery shaft 204. In addition, the delivery shaft 204 can be configured to be axially movable relative to the sleeve shaft 220 and the pusher shaft 212. As described further below, a distal end of the pusher shaft 212 can be inserted into a lumen of the sleeve shaft 220 and press against the proximal end of the docking device 52 retained inside the sleeve shaft 220.

After reaching a target implantation site, the docking device 52 can be deployed from the delivery shaft 204 by manipulating the pusher shaft 212 and sleeve shaft 220 using a dock handle 218 (also referred to as a “second handle” or a “hub assembly”), as described further below. For example, by pushing the pusher shaft 212 in the distal direction while holding the delivery shaft 204 in place or retracting the delivery shaft 204 in the proximal direction while holding the pusher shaft 212 in place, or by pushing the pusher shaft 212 in the distal direction while simultaneously retracting the delivery shaft 204 in the proximal direction, the docking device 52 can be pushed out of a distal end 204d of the delivery shaft 204, thus permitting the docking device 52 to transition from a delivery configuration to a deployed configuration (see FIG. 5). In certain examples, the pusher shaft 212 and the sleeve shaft 220 can be actuated independently of each other.

During delivery, the docking device 52 can be coupled to the delivery apparatus 200 via a release suture 222 (see FIG. 6B), or other retrieval line comprising a string, yarn, or other material that can be configured to be tied around the docking device 52 and cut for removal, that extends through the pusher shaft 212. In one specific example, the release suture 222 can extend through the delivery apparatus 200, e.g., through an inner lumen of the pusher shaft 212, to a suture lock assembly 216 of the delivery apparatus 200.

The handle assembly 202 can further include one or more flush ports (e.g., flush port 232 is shown in FIG. 6A, flush port 234 is shown in FIGS. 6A-6B) to supply flush fluid to one or more lumens arranged within the delivery apparatus 200 (e.g., annular lumens arranged between coaxial components of the delivery apparatus 200), for example, to maintain hemostasis within the delivery apparatus 200.

Further details on delivery apparatus/catheters/systems (including various examples of the handle assembly) that are configured to deliver a docking device to a target implantation site can be found in International Application No. PCT/US2020/036577 and in U.S. Patent Publication Nos. 2018/0318079 and 2018/0263764, each of which is incorporated by reference herein in its entirety.

As introduced above, the handle assembly 202 can further include a dock handle 218 to which the suture lock assembly 216 and a sleeve handle 224 are attached. The dock handle 218 can be configured to independently control the pusher shaft 212 and the sleeve shaft 220. The sleeve handle 224 is coupled to a proximal end of the sleeve shaft 220 and can control an axial position of the sleeve shaft 220 relative to the pusher shaft 212. In this way, operation of the various components of the handle assembly 202 can actuate and control operation of the components arranged within the delivery shaft 204. In some examples, the dock handle 218 can be coupled to the main handle 206 via a connector 226.

FIG. 6B shows an example of the dock handle 218 of the handle assembly 202 in more detail. In some examples, as shown, the dock handle 218 can include a Y-shaped connector 228 (also referred to as an “adaptor”) having a straight section 230 (e.g., straight conduit) and at least one branch 236 (e.g., branch conduit), although, in some examples, it can include more than one branch.

The dock handle 218 can be adapted and configured to allow a proximal segment 238 of the pusher shaft 212 (or another, similar pusher shaft) to extend to the suture lock assembly 216 arranged at the end of the branch 236, while a proximal portion 240 of the sleeve shaft 220 extends to the sleeve handle 224, arranged at the proximal end of the straight section 230 (see also FIG. 7). With this configuration, a medical professional can execute the deployment of the docking device (e.g., docking device 52 of FIG. 5) by manipulating the position of the dock handle 218 (e.g., moving it in the axial direction) and also execute retraction of the sleeve shaft 220 (off of and away from the implanted docking device) by pulling back, in the axial direction, on the sleeve handle 224.

In this way, the sleeve shaft 220 and pusher shaft 212 can be configured to work together such that they can be moved simultaneously together when deploying and positioning the docking device at the native valve (e.g., by moving the entire dock handle 218 forward and/or backward, in the axial direction), but can also to move independently so the pusher shaft 212 can hold the docking device in position while the sleeve shaft 220 is retracted off of the docking device (e.g., by holding the dock handle 218 in place relative to the outer shaft 204 of the delivery apparatus 200 and/or other parts of the delivery apparatus 200 and/or docking device while pulling proximally on the sleeve handle 224 to withdraw the sleeve shaft 220).

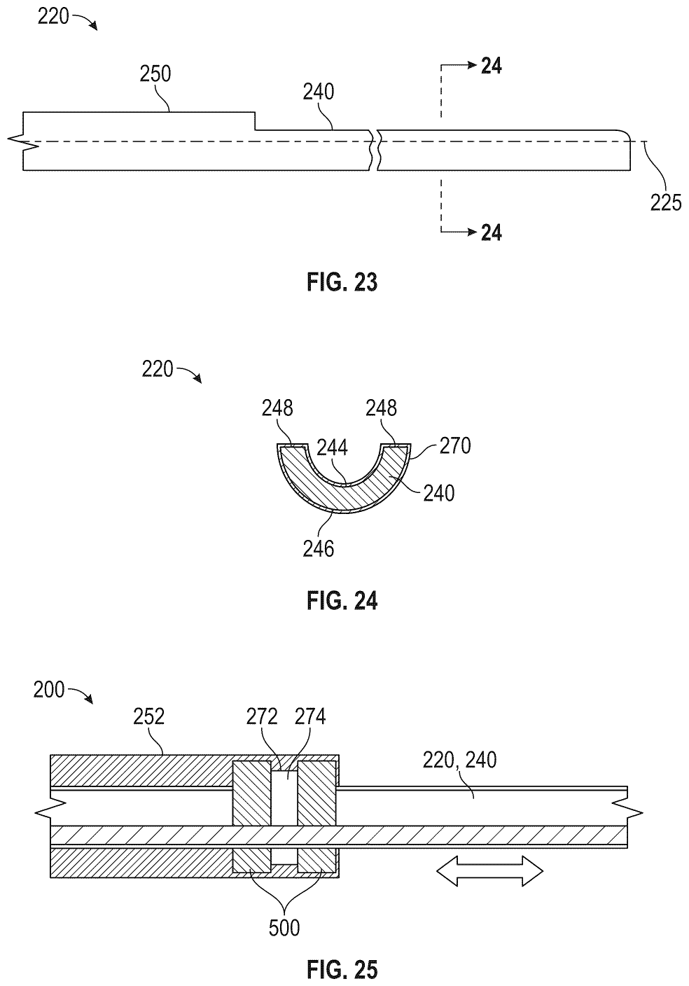

As shown in FIG. 7, the proximal portion 240 of sleeve shaft 220 has an outer surface 242 that includes an inwardly-facing surface 244 and an outwardly-facing surface 246. The inwardly-facing surface 244 can define an open channel. For example, the channel of the sleeve shaft 220 is open in a radial direction, such that the proximal segment 238 of the pusher shaft 212 can extend out of the open channel and away from the sleeve shaft 220 at an angle relative to a longitudinal axis 225 (FIG. 23) of the sleeve shaft 220 (e.g., through the branch 236). In some examples, the proximal portion 240 of the sleeve shaft 220 can also be referred to herein as an “open channel 240.”

As shown in FIG. 7, the open channel 240 can have a generally U or C-shaped cross-section. In some examples, as depicted, the outer surface 242 is curved, such that the open channel 240 has a partially annular cross-section (e.g., a C-shaped cross-section). Specifically, the open channel 240 can be partially annular such that the inwardly-facing surface 244 is a concave surface and the outwardly-facing surface 246 is a convex surface. Edge surfaces or edges 248 can define a junction between the concave (or inwardly-facing) surface 244 and the convex (or outwardly-facing) surface 246. In this way, the inwardly-facing surface 244 of the open channel 240 can form a void space in which the pusher shaft 212 can be at least partially disposed (FIG. 7). In various examples, the open channel 240 can be cut using a laser (e.g., to form the edges 248, etc.), although any other means for forming the open channel (e.g., removing part of the tubular structure) can be used.

A distal segment 250 of the sleeve shaft 220 can comprise a closed channel or lumen, such that the channel is closed in the radial direction (e.g., an annular cross-section) (FIG. 6A). The pusher shaft 212 can extend through the closed channel of the distal segment 250. For example, the pusher shaft 212 can be coaxial with the sleeve shaft 220 along some or a majority of the delivery apparatus 200, such as through the distal segment 250 of the sleeve shaft 220. The distal segment 250 can extend from a distal end of the sleeve shaft 220 to the open channel 240, for example, at an intermediate axial location of the sleeve shaft 220. The open channel 240 of the sleeve shaft 220 can extend from the intermediate axial location of the sleeve shaft 220 to the proximal end of the sleeve shaft 220, for example, to the sleeve handle 224. In other examples, the open channel 240 of the sleeve shaft 220 can extend proximally from the intermediate axial location without extending to the proximal end of the sleeve shaft 220. In these examples, the open channel 240 can form an axially-extending window or slot that permits the proximal segment 238 of the pusher shaft 212 to extend out and way from the sleeve shaft 220 at an angle.

The dock handle 218 can include a seal housing 252, for example, disposed at a proximal end portion of the straight section 230. In some examples, a housing of the dock handle 218 can include the seal housing 252 or define a portion of the seal housing 252. In some examples, as depicted in FIG. 15, the seal housing 252 can be separately formed from the housing of the dock handle 218 and coupled thereto.

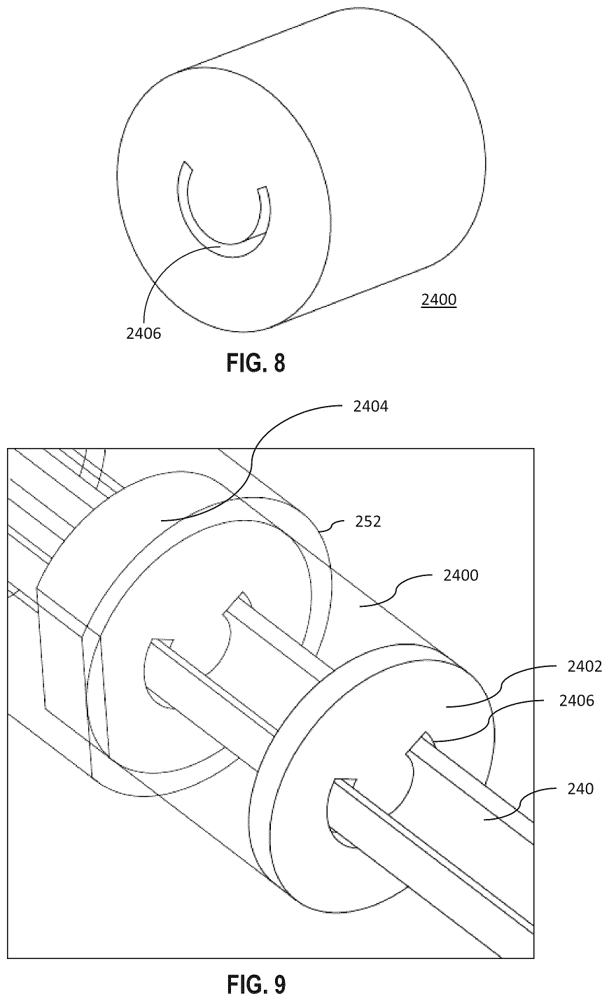

The seal housing 252 can house various gaskets, seals, and/or washers to form a seal around the open channel 240 of the sleeve shaft 220. For example, FIGS. 8 and 9 illustrate an example of a hemostatic seal 2400 that can be disposed within the seal housing 252 and used to seal around the open channel 240 of the sleeve shaft 220. In some examples, as depicted in FIGS. 6A-6B, a locking knob 254 can be coupled to the seal housing 252, for example, via a threaded connection. The locking knob 254 can be transitioned between an active or locked configuration and an inactive or unlocked configuration. In the locked configuration, the locking knob 254 can be configured to lock the sleeve shaft 220 such that the sleeve shaft 220 is prevented from moving relative to the dock handle 218, as well as to apply a sufficient pressure to the hemostatic seal 2400 to actively seal the open channel 240 of the sleeve shaft 220. In the unlocked configuration, this pressure is removed from the hemostatic seal 2400 such that the sleeve shaft 220 is permitted to move relative to the dock handle 218. In some instances, in the unlocked configuration, the seal 2400 may not be actively sealing the sleeve shaft 220 and/or not providing hemostasis. In this way, the hemostatic seal 2400 can be considered an active seal.

As seen in FIG. 8, the hemostatic seal 2400 can possess an opening 2406 in the shape of a cross-section of the open channel 240 of the sleeve shaft 220, such as a U or C-shape or incomplete (e.g., partial) annulus, configured to receive the open channel 240 therein and to seal on all sides of the sleeve shaft 220 (e.g., surfaces 244, 246 and edges 248). FIG. 9 illustrates an example of the hemostatic seal 2400 as arranged within a portion of the seal housing 252. In FIG. 9, the seal 2400 and the portion of the seal housing 252 are transparent and the locking knob 254 is omitted for illustration purposes. In some examples, as shown in FIG. 9, two rigid washers 2402 and 2404 can support each end of the hemostatic seal 2400. The rigid washers 2402, 2404 can possess the same profile as the hemostatic seal 2400 to maintain the integrity of the hemostatic seal 2400. In the locked configuration, the rigid washers 2402, 2404 can place inward pressure on the hemostatic seal 2400 to ensure a seal between the hemostatic seal 2400 and the open channel 240 of the sleeve shaft 220, based on contact between the locking knob 254 and the proximal washer 2402.

In some examples, it can be desirable for the sleeve shaft 220 to be sealed independent of a locked state of the sleeve shaft 220. For example, rather than a seal that is selectively active (e.g., actively sealing in the locked configuration), it can be desirable for the sleeve shaft 220 to be sealed by a passive seal that enables a hemostatic seal around the sleeve shaft 220 while the sleeve shaft 220 is free to move relative to the handle assembly 202. In other words, a passive hemostatic seal can exist around the sleeve shaft 220 regardless of whether the sleeve shaft 220 is locked or unlocked. In these examples, a locking mechanism can be used to fix the sleeve shaft 220 relative to the handle assembly 202 that operates independently of the hemostatic seal for the sleeve shaft 220. For example, the locking mechanism can include structure other than locking knob 254, such as a collet, a clamp, etc. configured to apply a frictional and/or compressive force to the sleeve shaft 220 to prevent movement of the sleeve shaft 220 relative to the handle assembly 202. In some examples, the locking mechanism can be spaced apart from and/or proximal to a seal for the sleeve shaft 220. Additional examples of locking mechanisms are described in U.S. Provisional Patent Application No. (Atty. Docket No. THVDL-13027US01), which is incorporated by reference herein in its entirety.

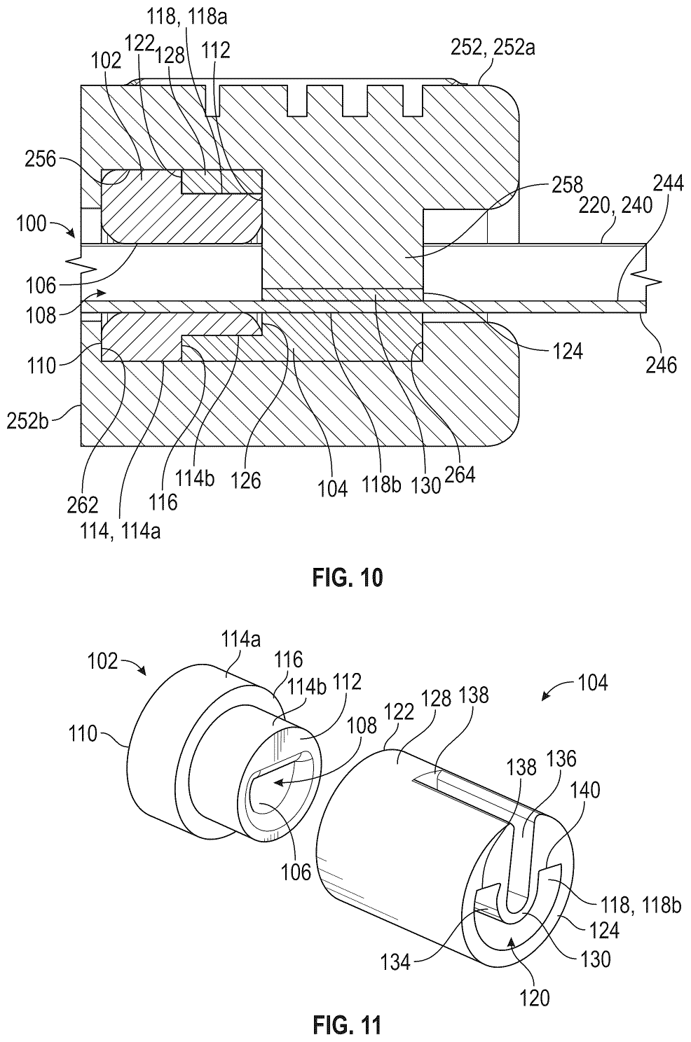

FIGS. 10-15 illustrate an example of a hemostatic seal 100 that passively seals the open channel 240 of the sleeve shaft 220 and can be disposed within the seal housing 252. For example, the seal 100 can provide hemostasis during relative movement between the sleeve shaft 220 and the seal 100. The passive hemostatic seal 100 can provide a sufficient sealing force to the sleeve shaft 220, for example, based on a compressive force applied to the seal 100 (e.g., by the seal housing 252, etc.), independent of a locking mechanism for the sleeve shaft 220.

As shown, the hemostatic seal 100 includes multiple sealing members to seal around the open channel 240 within the seal housing 252. Specifically, the hemostatic seal 100 includes a first (or external) sealing member 102 and a second (or internal) sealing member 104 that is coupled to the external sealing member 102. The external sealing member 102 and the internal sealing member 104 are coupled together in a manner that forms a seal therebetween. The external sealing member 102 can be configured to seal around at least the outwardly-facing surface 246 of the open channel 240. For example, the external sealing member 102 can seal a first gap between the outwardly-facing surface 246 and an inner wall 256 of the seal housing 252. The internal sealing member 104 can be configured to seal against at least the inwardly-facing surface 244 of the open channel 240. For example, the internal sealing member 104 can seal a second gap between the inwardly-facing surface 244 and the inner wall 256 of the seal housing 252.

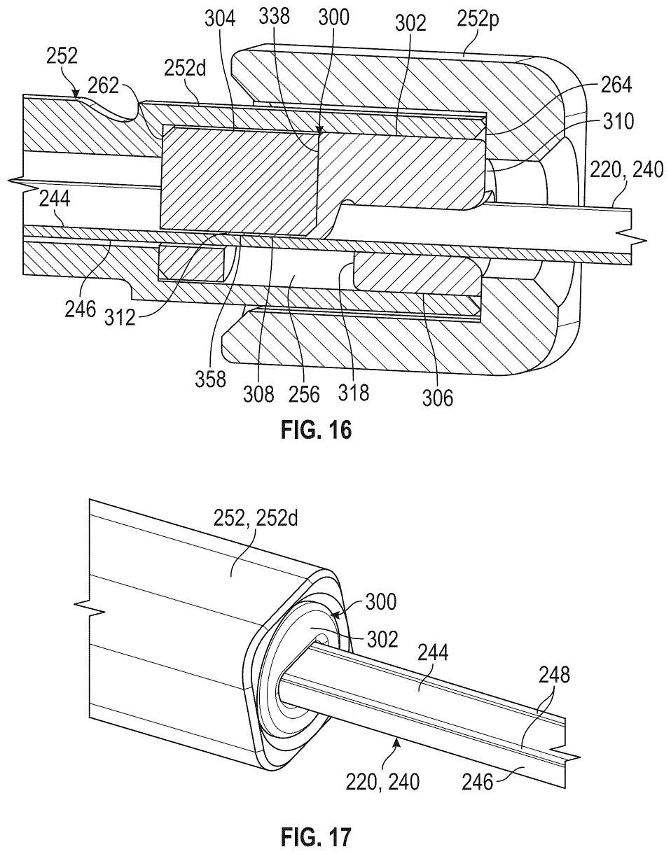

In some instances, the seal housing 252 can be integrally formed as a single, unitary component. In other instances, as depicted in FIGS. 10 and 14-15, the seal housing 252 can comprise one or more segments that are formed as separate components that are coupled together (e.g., via fasteners, adhesive, mating features, and/or other means for coupling). For example, the seal housing 252 can comprise a first or upper segment 252a and a second or lower segment 252b that are coupled together via mating features (e.g., a pin 260 and socket connection (FIG. 15)). In other examples, as depicted in FIGS. 16-17, the seal housing 252 can include a distal segment 252d and a proximal segment 252p (e.g., instead of the upper and lower segments 252a, 252b). In some examples, the seal housing 252 can be manufactured using one or more molding processes (e.g., injection molding, etc.).

The inner wall 256 can at least partially define a chamber in which the hemostatic seal 100 can be disposed. When the hemostatic seal 100 is disposed within the chamber of the seal housing 252 and around the sleeve shaft 220, the hemostatic seal 100 creates a passive, hemostatic seal around the sleeve shaft 220. For example, the seal housing 252 can provide sufficient compression to the hemostatic seal 100 such that the open channel 240 of the sleeve shaft 220 is sealed while the sleeve shaft 220 is free to move relative to the dock handle 218. In addition to the inner wall 256, the chamber of the seal housing 252 can be defined by one or more inner surfaces of the seal housing 252 including a distal surface 262 and a proximal surface 264 (FIG. 10). As shown, the hemostatic seal 100 can be positioned between the distal and proximal surfaces 262, 264 and compressed within the chamber of the seal housing 252 (e.g., by the inner wall 256, by the wedge 258, and/or by the distal and proximal surfaces 262, 264). In this way, the hemostatic seal 100 creates a passive hemostatic seal around the sleeve shaft 220, including while the sleeve shaft 220 is moving relative to the seal 100, the seal housing 252, and/or the dock handle 218 (e.g., independent of a locked or unlocked state of the sleeve shaft 220).

The external sealing member 102 includes an inner surface 106 that defines an opening 108 extending through the sealing member 102. The opening 108 extends from a first end 110 to a second end 112 of the sealing member 102 and the sleeve shaft 220 can extend through the opening 108. In some examples, as depicted, the inner surface 106 can include a first or flat portion 106a and a second or curved portion 106b (FIG. 13A), such that the opening 108 is D-shaped. As shown, the second portion 106b can generally correspond to and complement the shape of the outwardly-facing surface 246. For example, the second portion 106b can be an inwardly-facing surface (e.g., concave curvature, etc.). When the external sealing member 102 is disposed around the sleeve shaft 220, the second portion 106b can contact the outwardly-facing surface 246 of the open channel 240 and have the same or substantially same radius of curvature as the outwardly-facing surface 246. In this way, the curved portion 106b can seal against and/or around the outwardly-facing surface 246 when the sleeve shaft 220 is disposed within the opening 108. In some examples, the flat portion 106a can contact the edges 248 of the open channel 240 and seal against and/or around the edges 248. As such, the external sealing member 102 can create a seal around the outer surfaces of the open channel 240 (e.g., the outwardly-facing surface 246 and, in some examples, the edges 248). In other examples, the inner surface 106 can define an opening having other shapes that engage with at least the outwardly-facing surface 246 of the sleeve shaft 220, including a circular opening, a square or rectangular opening (e.g., for u-shaped sleeve shafts 220), etc.

In some examples, the external sealing member 102 is generally cylindrical with a stepped outer surface 114. The outer surface 114 can include a first outer surface portion 114a adjacent to the first end 110 and a second outer surface portion 114b adjacent to the second end 112. An outer diameter of the first outer surface portion 114a can be sized to fit within the chamber of the seal housing 252, such that the external sealing member 102 contacts the inner wall 256 of both the upper segment 252a and the lower segment 252b. In this way, the first outer surface portion 114a can seal the first gap between the inner wall 256 of the seal housing 252 and the outwardly-facing surface 246 of the sleeve shaft 220.

An outer diameter of the second outer surface portion 114b can be smaller than an outer diameter of the first outer surface portion 114a. Specifically, the outer surface 114 of the external sealing member 102 includes a shoulder 116 axially between the first end 110 and the second end 112 that defines a transition between the first outer surface portion 114a and the second outer surface portion 114b.

The internal sealing member 104 includes an inner surface 118 defining an opening 120 through which the sleeve shaft 220 can extend. In some examples, the inner surface 118 can be stepped and have a first inner surface portion 118a adjacent to a first end 122 of the internal sealing member 104 and a second inner surface portion 118b adjacent to a second end 124 of the internal sealing member 104. An inner diameter of the first inner surface portion 118a can be sized to receive the second outer surface portion 114b of the external sealing member 102, such that the first inner surface portion 118a of the internal sealing member 104 contacts the second outer surface portion 114b of the external sealing member 102 when the sealing members 102, 104 are coupled together. In some examples, the first inner surface portion 118a can define a larger opening than the second inner surface portion 118b. For example, the radius of curvature of the second inner surface portion 118b can be smaller than the radius of curvature of the first inner surface portion 118a. In the illustrated example, the radius of curvature of the second inner surface portion 118b can be equal or approximately equal to a radius of curvature of the outwardly-facing surface 246 of the open channel 240. The inner surface 118 of the internal sealing member 104 can include a lip 126 positioned axially between the first end 122 and the second end 124 that defines a transition between the first inner surface portion 118a and the second inner surface portion 118b.

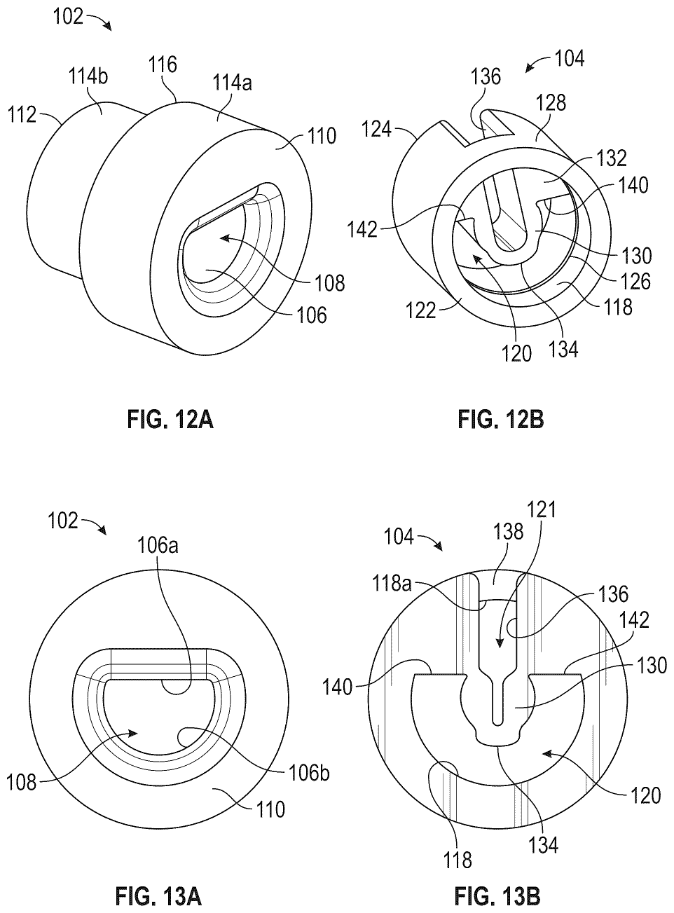

As introduced above, the external sealing member 102 and the internal sealing member 104 can be coupled or mated together such that a seal is formed between the sealing members 102, 104. For example, the sealing members 102, 104 can partially overlap in the radial and axial directions. In some examples, as depicted, the internal sealing member 104 can be disposed partially around the external sealing member 102. The internal sealing member 104 can include an extension 128 that projects axially outwards at the first end 122 of the internal sealing member 104 and extends over the external sealing member 102. The extension 128 can surround the second outer surface portion 114b of the external sealing member 102 and abut the shoulder 116. Specifically, the first inner surface portion 118a (an inner surface of the extension 128) can contact the second outer surface portion 114b when the external sealing member 102 and the internal sealing member 104 are mated together. As shown in FIG. 12B, the extension 128 and the first inner surface portion 118a have a shape (e.g., annular) corresponding to the shape of the second outer surface portion 114b of the sealing member 102.

The second end 112 of the external sealing member 102 can contact the lip 126 and/or an inner projection 130 of the internal sealing member 104. In some examples, as depicted, the lip 126 and the inner projection 130 can define a first intermediate surface 132 (FIG. 12B) that is parallel to the first end 122 of the internal sealing member 104. The first intermediate surface 132 is disposed axially between the first end 122 and the second end 124 of the internal sealing member 104. When the external sealing member 102 and the internal sealing member 104 are mated together, the second end 112 of the external sealing member 102 can contact the first intermediate surface 132 and the first end 122 of the internal sealing member 104 can contact the shoulder 116 of the external sealing member 102. For example, the axial distance between the first intermediate surface 132 and the first end 122 of the internal sealing member 104 can be equal to the axial distance between the shoulder 116 and the second end 112 of the external sealing member 102. In other words, the first inner surface portion 118a of the internal sealing member 104 and the second outer surface portion 114b of the external sealing member 102 can be the same length.

The lip 126 can define a step along the inner surface 118 of the internal sealing member 104. In this way, the first inner surface portion 118a can contact the second outer surface portion 114b of the external sealing member 102 and the second inner surface portion 118b can contact the outwardly-facing surface 246 of the open channel 240, when the sleeve shaft 220 is disposed within the hemostatic seal 100. In some examples, a height of the lip 126 can be smaller (e.g., smaller than the height is shown in FIG. 10) or the lip 126 can be omitted, such that a portion of the inner surface of the internal sealing member 104 does not contact the sleeve shaft 220 (e.g., relying on the external sealing member 102 for sealing of the gap between the seal housing 252 and the outwardly-facing surface 246 of the sleeve shaft 220).

In some examples, the sealing members 102, 104 can be mated together in other manners and/or using different structures than the shoulder 116 and the extension 128. For example, rather than the external sealing member 102 having a reduction in outer diameter size to fit within the internal sealing member 104, the internal sealing member 104 can be configured to fit within the opening 108 of the external sealing member 102. Specifically, the outer surface 114 of external sealing member 102 can have a continuous outer diameter and the inner surface 106 defining the opening 108 can be stepped, such that an extension of the internal sealing member 104 can be disposed within the opening 108 and abut a shoulder or lip therein. Accordingly, in some examples, the external sealing member 102 can be disposed partially around the internal sealing member 104.

In some examples, as shown, the first end 110 of the external sealing member 102 can contact the distal surface 262 of the seal housing 252 and the second end 124 of the internal sealing member 104 can contact the proximal surface 264 of the seal housing 252. In other examples, the hemostatic seal 100 can be oriented differently, such that the first end 110 of the external sealing member 102 can contact the proximal surface 264 and the second end 124 of the internal sealing member 104 can contact the distal surface 262.

The sealing members 102, 104 can have a lower durometer hardness than the seal housing 252 and the sleeve shaft 220. For example, the sealing members 102, 104 can be relatively softer and/or more flexible than the seal housing 252 and sleeve shaft 220, such that the sealing members 102, 104 can be compressed against the inner surfaces of the seal housing 252 and around the surfaces of the sleeve shaft 220 to create a hemostatic seal around the sleeve shaft 220 within the seal housing 252.

The inner projection 130 of the internal sealing member 104 can be configured to seal against the inwardly-facing surface 244 of the open channel 240. For example, the inner projection 130 can extend into the opening 120 of the internal sealing member 104 in a direction perpendicular to a longitudinal axis of the internal sealing member 104 (and perpendicular to a longitudinal axis 225 of the sleeve shaft 220). When the sleeve shaft 220 is disposed within the opening 120, an engaging surface 134 of the inner projection 130 can contact the inwardly-facing surface 244 of the open channel 240. As shown, the engaging surface 134 can generally correspond to and complement the shape of the inwardly-facing surface 244. For example, the engaging surface 134 can have an outwardly-facing surface (e.g., convex curvature, etc.). In this way, the inner projection 130 can extend or project into the opening defined by the open channel 240 and the engaging surface 134 can seal against the inwardly-facing surface 244.

To ensure a sufficient seal between the engaging surface 134 of the inner projection 130 and the inwardly-facing surface 244 of the sleeve shaft 220, the inner projection 130, or a portion thereof, can comprise a different material than the rest of the internal sealing member 104 and/or the external sealing member 102. In some examples, the material can be stiffer and/or more rigid to reinforce the structural integrity of the inner projection. In some examples, the material can be a material having a lower durometer hardness (e.g., more compressible) to offer better sealing ability.

In some examples, to ensure a sufficient seal between the engaging surface 134 of the inner projection 130 and the inwardly-facing surface 244 of the sleeve shaft 220, a more rigid component can be at least partially positioned within the inner projection 130. For example, a relatively hard or rigid component can reinforce the structural integrity of the inner projection 130 such that the inner projection 130 applies a sufficient sealing force to the inwardly-facing surface 244. In some examples, a separate structure or component can be disposed within an opening or slot of the inner projection 130.

To accommodate a relatively rigid component, the inner projection 130 can include a slot 136 that extends along the axial length of the inner projection 130. For example, the slot 136 can extend from the second end 124 to a second intermediate surface 138 (FIGS. 11 and 13B) disposed axially between the first and second ends 122, 124 of the internal sealing member 104. As shown, the second intermediate surface 138 is located at or adjacent to the lip 126.

In some examples, at least a portion of the seal housing 252 can extend or project radially inwards from the inner wall 256. When the internal sealing member 104 is positioned within the seal housing 252, this portion of the seal housing 252 can extend into the slot 136. For example, the seal housing 252 can include a projection or wedge 258 that is configured to extend into the slot 136 and can ensure the internal sealing member 104 contacts and/or is expanded against the inwardly-facing surface 244 of the open channel 240. Prior to positioning of the wedge 258 within the slot 136, components (e.g., the sleeve shaft 220) can be free to move relative to the seal 100, which can offer advantages to the assembly process. After components are positioned relative to the seal 100, the seal 100 can be activated by positioning the wedge 258 within the slot 136 (e.g., at the end of the assembly process). As shown in FIG. 10, the upper segment 252a can include the wedge 258 (see also FIG. 15). In other examples, other segments of the seal housing 252 (e.g., a distal segment 252d of the seal housing 252, etc.) can include the wedge 258.

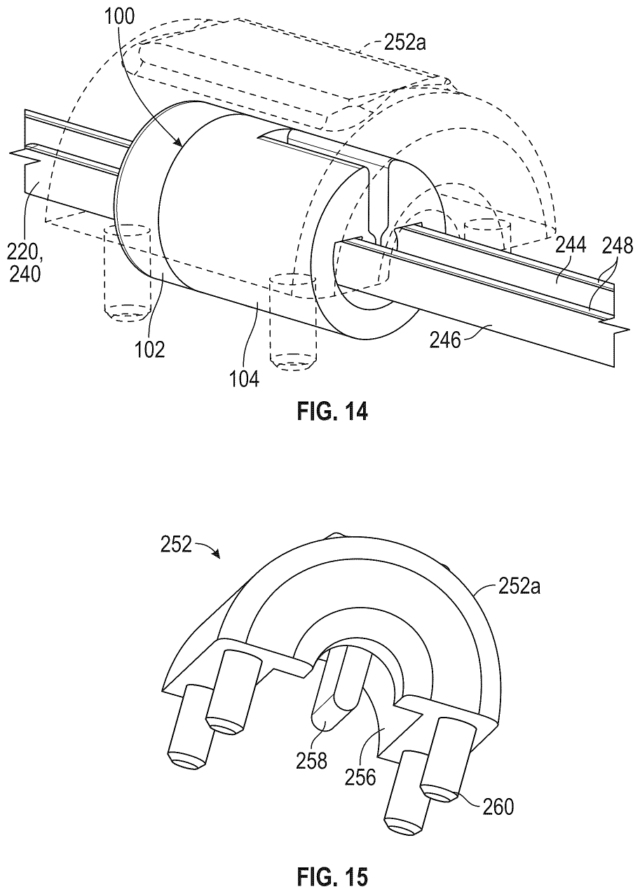

As shown in FIG. 13B, which shows an end view of the internal sealing member 104, the inner projection 130 defines two openings that extend axially through the internal sealing member 104. The open channel 240 can extend through the lower opening 120 and the wedge 258 can extend radially into an upper opening 121 defined by the slot 136. Specifically, when the internal sealing member 104 is disposed within the seal housing 252, the wedge 258 is positioned with the upper opening 121 and contacts the slot 136. In this way, the wedge 258 can be configured to hold the inner projection 130 in place relative to the seal housing 252 to ensure a seal between the engaging surface 134 of the inner projection 130 and the inwardly-facing surface 244 of the open channel 240. In some examples, the shape of the slot 136 can corresponds to the shape of the wedge 258 (e.g., have a constant width; see FIGS. 11, 12B). In other examples, as shown in FIGS. 13B and 14, the slot 136 can be tapered or stepped, such that the slot 136 is wider at a location closer to the outer surface of the internal sealing member 104 and is narrower towards the lower end of the slot 136. In these examples, the wedge 258 can stretch or expand the narrow portion of the slot 136 radially outwards against the inwardly-facing surface 244 of the open channel 240.

The slot 136 is shown as an axial through slot, such that the first intermediate surface 132 and the second end 124 of the internal sealing member 104 define open ends of the slot 136. In some examples, rather than the slot 136 being a through-slot having an opening 121 that extends through the first intermediate surface 132 and the second end 124, the slot 136 can extend less than the entire axial length of the inner projection 130. For example, ends of the slot 136 can be spaced apart from the first intermediate surface 132 and/or the second end 124 of the internal sealing member 104, such that the slot 136 is only open in the radial direction, through the outer surface of the internal sealing member 104.

The inner projection 130 can also be configured to seal against the edges 248 of the open channel 240. For example, the inner projection 130 can include flat, axially-extending surfaces 140, 142 disposed on either side of the engaging surface 134 that seal against the edges 248. The second inner surface portion 118b, the engaging surface 134 and the surfaces 140, 142 can define the opening 120 at the second end 124 of the internal sealing member 104. At the first end 122 of the internal sealing member 104, the opening 120 is defined by the first inner surface portion 118a. In this way, the opening 120 has a different shape at the first end 122 of the internal sealing member 104 (e.g., corresponding to the shape of the second end 112 of the external sealing member 102) than at the second end 124 of the internal sealing member 104 (e.g., corresponding to the shape of the open channel 240 of the sleeve shaft 220).

In some examples, a passive hemostatic seal can include one sealing member, rather than multiple sealing members. FIGS. 16-20 illustrate an example of a hemostatic seal 300 that passively seals the open channel 240 of the sleeve shaft 220 and can be disposed within the seal housing 252. As shown, the hemostatic seal 300 includes a sealing member 302 to seal around the open channel 240 and a seal block 304 that is coupled to the sealing member 302. The sealing member 302 can be configured to seal the open channel 240 within the chamber of the seal housing 252 (e.g., between the open channel 240 and the inner wall 256). The seal block 304 can be configured to expand and/or compress a portion of the sealing member 302 against at least the inwardly-facing surface 244 of the open channel 240, for example, similar to the wedge 258 of the seal housing 252.

In some examples, the seal block 304 can be used instead of and/or in addition to the wedge 258. For example, as illustrated in FIGS. 16-17, the seal housing 252 does not include the wedge 258. As introduced above, in some examples, the seal housing 252 can comprise a first or distal segment 252d and a second or proximal segment 252p that is coupled to the first segment 252d. For example, the second segment 252p can be a cap that is fitted around a proximal end of the first segment 252d and secured thereto. In the illustrated example, the first segment 252d includes the distal surface 262 and the inner wall 256, and the second segment 252p includes the proximal surface 264.

As shown, the hemostatic seal 300 can be positioned between the distal and proximal surfaces 262, 264 and compressed within the chamber of the seal housing 252 (e.g., by the inner wall 256 and/or by the distal and proximal surfaces 262, 264). In this way, the hemostatic seal 300 creates a passive hemostatic seal around the sleeve shaft 220, including while the sleeve shaft 220 is moving relative to the seal 300, the seal housing 252, and/or the dock handle 218 (e.g., independent of a locked or unlocked state of the sleeve shaft 220). While the hemostatic seal 300 is shown as positioned within a seal housing 252 having a distal segment and a proximal segment, the hemostatic seal 300 can be disposed within any seal housing (e.g., any seal housing described herein, a seal housing having upper and lower segments, etc.).

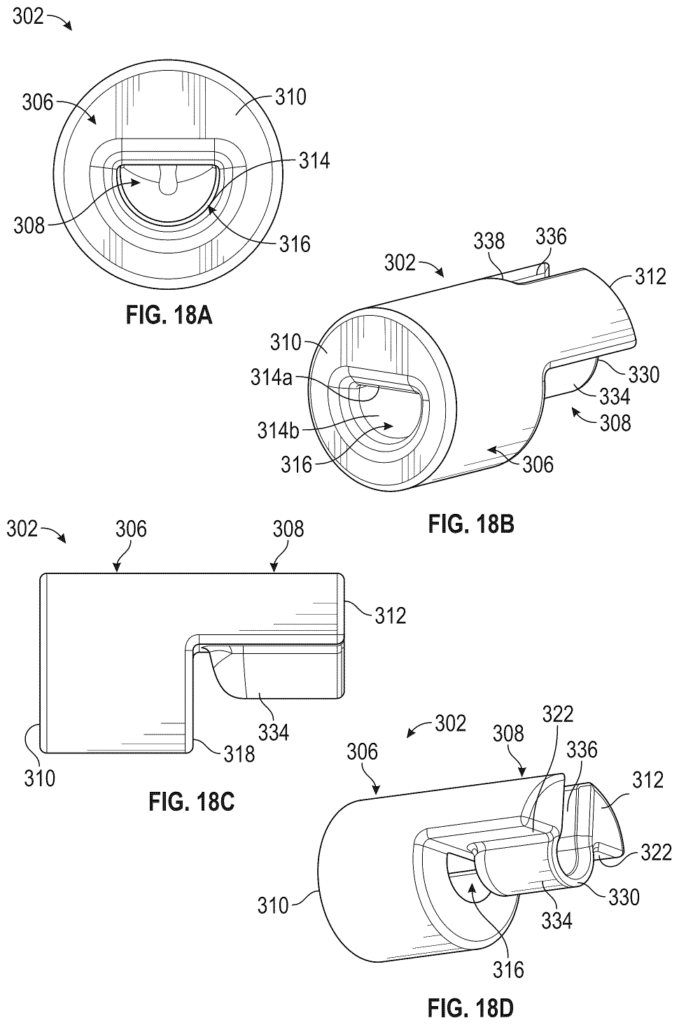

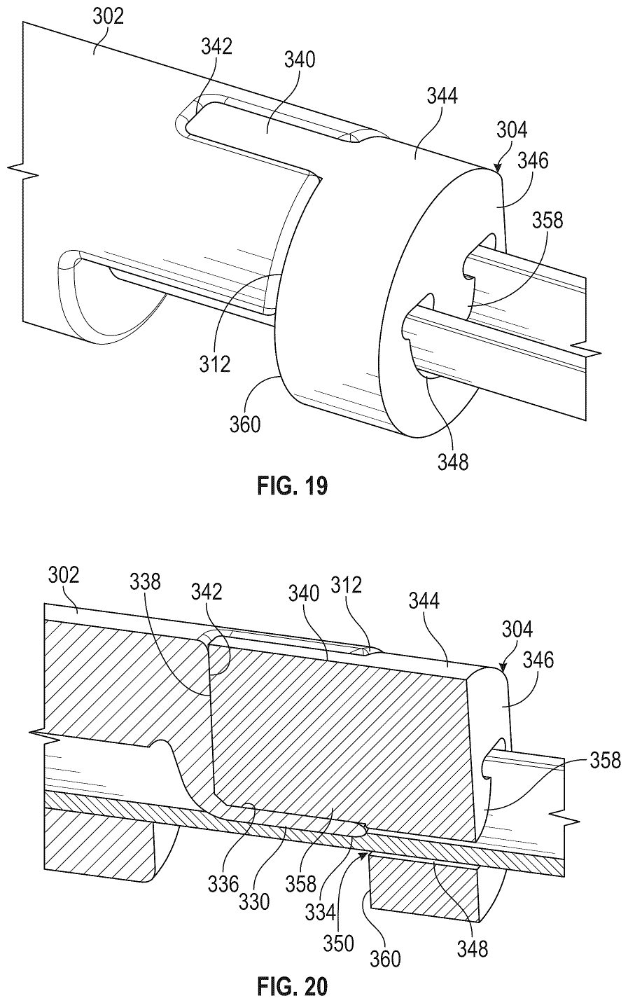

FIG. 16 illustrates a cross-sectional view of the hemostatic seal 300 disposed within the chamber of the seal housing 252. FIG. 17 illustrates the hemostatic seal 300 within the seal housing 252 with the second segment 252p removed for purposes of illustration. FIGS. 18A-18D show multiple views of the sealing member 302. FIGS. 19-20 show the hemostatic seal 300 disposed around the open channel 240 of the sleeve shaft 220.

The sealing member 302 is generally cylindrical and includes a first or external sealing portion 306 and a second or internal sealing portion 308. The external sealing portion 306 is adjacent to a first end 310 of the sealing member 302 and is configured to seal around and/or against at least the outwardly-facing surface 246 of the open channel 240. For example, the external sealing portion 306 can seal a first gap between the outwardly-facing surface 246 and the inner wall 256 of the seal housing 252. The internal sealing portion 308 is adjacent to the second end 312 of the sealing member 302 and is configured to seal around and/or against at least the inwardly-facing surface 244 of the open channel 240. For example, the internal sealing portion 308 can seal a second gap between the inwardly-facing surface 244 and the inner wall 256 of the seal housing 252.

As shown in FIGS. 18A-18D, the external sealing portion 306 includes an inner surface 314 defining an opening 316 that extends through the external sealing portion 306 from the first end 310 to a first intermediate surface 318 positioned axially between the first and second ends 310, 312. As shown, the first intermediate surface 318 is parallel to the surface of the first end 310 of the sealing member 302 (e.g., perpendicular to a longitudinal axis of the sealing member 302). In some examples, as depicted, the inner surface 314 can include a first flat portion 314a and a second or curved portion 314b (see FIG. 18B), such that the opening 108 is D-shaped. As shown, the second portion 314b can generally correspond to and complement the shape of the outwardly-facing surface 246. For example, the second portion 314b can be an inwardly-facing surface (e.g., concave curvature, etc.). When the sealing member 302 is disposed around the sleeve shaft 220, the curved portion 314b can contact the outwardly-facing surface 246 of the open channel 240 and have the same or substantially same radius of curvature as the outwardly-facing surface 246. In this way, the curved portion 314b can seal against and/or around the outwardly-facing surface 246 when the sleeve shaft 220 is disposed within the opening 316. In some examples, the flat portion 314a can contact the edges 248 of the open channel 240 and seal against and/or around the edges 248. As such, the external sealing portion 306 can create a seal around the outer surfaces of the open channel 240 (e.g., the outwardly-facing surface 246 and, in some examples, the edges 248). In other examples, the inner surface 314 can define an opening having other shapes that engage with at least the outwardly-facing surface 246 of the sleeve shaft 220, including a circular opening, a square or rectangular opening (e.g., for u-shaped sleeve shafts 220), etc.

In some examples, the sealing member 302 is generally cylindrical. For example, an outer diameter of the external sealing portion 306 can be sized to fit within the chamber of the seal housing 252, such that an outer surface of the external sealing portion 306 contacts the inner wall 256 of the seal housing 252. In some examples, as illustrated, the external sealing portion 306 contacts the inner wall 256 within the distal segment 252d and contacts the proximal surface 264 within the proximal segment 252p. In this way, the external sealing portion 306 can seal the first gap between the inner wall 256 of the seal housing 252 and the outwardly-facing surface 246 of the sleeve shaft 220.

In some examples, as shown in FIG. 16, the first end 310 of the sealing member 302 can contact the proximal surface 264 of the seal housing 252 and the second end 312 of the sealing member 302 can be positioned towards the distal surface 262 of the seal housing 252. The seal block 304 can contact the distal surface 262. In other examples, the hemostatic seal 300 can be oriented differently, such that the first end 310 of the sealing member 102 can contact the distal surface 262 and the second end 312 of the sealing member 302 is positioned towards the proximal surface 264. The seal block 304 can contact the proximal surface 264.

The sealing member 302 can have a lower durometer hardness than the seal housing 252, the sleeve shaft 220, and the seal block 304. For example, the sealing member 302 can be relatively softer and/or more flexible than the seal housing 252, the sleeve shaft 220, and the seal block 304, such that the seal block 304 can compress at least a portion of the sealing member 302 against a surface of the sleeve shaft 220 and the sealing member 302 can be compressed against the inner surfaces of the seal housing 252 and around the surfaces of the sleeve shaft 220 to create a hemostatic seal around the sleeve shaft 220 within the seal housing 252.

The internal sealing portion 308 can include an inner projection 330 configured to seal against the inwardly-facing surface 244 of the open channel 240. For example, the inner projection 330 can extend radially inwards from an outer surface of the sealing member 302, for example, in a direction perpendicular to a longitudinal axis of the sealing member 302 (and perpendicular to a longitudinal axis 225 of the sleeve shaft 220). The inner projection 330 extends axially along a length of the internal sealing portion 308. When the hemostatic seal 300 is coupled to the sleeve shaft 220, the inner projection 330 extends axially along a length of the open channel 240. In this way, the inner projection 330 can form a flap or tongue that can be compressed against the inwardly-facing surface 244 of the open channel 240. Specifically, when the sleeve shaft 220 is disposed within the opening 316 of the sealing member 302, an engaging surface 334 of the inner projection 330 can contact the inwardly-facing surface 244 of the open channel 240. As shown, the engaging surface 334 can generally correspond to and complement the shape of the inwardly-facing surface 244. For example, the engaging surface 334 can have an outwardly-facing surface (e.g., convex curvature, etc.). In this way, the inner projection 330 can extend or project into the opening defined by the open channel 240 and the engaging surface 334 can seal against the inwardly-facing surface 244.

The internal sealing portion 308 can also be configured to seal against the edges 248 of the open channel 240. For example, the internal sealing portion 308 can include flat, axially-extending surfaces 322 disposed on either side of the inner projection 330 that seal against the edges 248.

To ensure a sufficient seal between the engaging surface 334 of the inner projection 330 and the inwardly-facing surface 244 of the sleeve shaft 220, a more rigid component can be at least partially positioned within the inner projection 330. For example, a relatively rigid component can reinforce the structural integrity of the inner projection 330 such that the inner projection 330 applies a sufficient sealing force to the inwardly-facing surface 244. In some instances, the inner projection 330, or a portion thereof, can comprise a different material (e.g., stiffer, more rigid, etc.) than the rest of the sealing member 302. In some examples, a separate structure or component can be disposed within an opening or slot of the inner projection 330.

To accommodate a relatively rigid component, the inner projection 330 can include a slot 336 that extends along a portion of the axial length of the inner projection 330. For example, the slot 336 can extend from the second end 312 to a second intermediate surface 338 (FIG. 18B) disposed axially between the first and second ends 310, 312 of the sealing member 302. As shown, the second intermediate surface 338 is axially spaced apart from the first intermediate surface 318. In other examples, the second intermediate surface 338 can be axially aligned with the first intermediate surface 318.

As described above, in some examples, a portion of the seal housing 252 (e.g., wedge 258) can extend into the slot 336. In some examples, both a portion of the seal housing 252 and a portion of the seal block 304 can extend into the slot 336. In other examples, as depicted in FIGS. 16 and 19-20, a portion of the seal block 304 can extend into the slot 336, instead of the seal housing 252. When the seal block 304 is coupled to the sealing member 302, this portion of the seal block 304 can extend into the slot 336. For example, the seal block 304 can include a projection or wedge 358 that is configured to extend into the slot 336 and can ensure the engaging surface 334 of the inner projection 330 contacts and/or is expanded against the inwardly-facing surface 244 of the open channel 240.

As shown in FIGS. 19 and 20, the seal block 304 can include a first portion 340 at a first end 342 of the seal block 304 that contacts the second intermediate surface 338 of the sealing member 302. The seal block 304 can include a second portion 344 at a second end 346 of the seal block 304. The second portion 344 can include an inner surface 348 that can define an opening 350. The open channel 240 can extend through the opening 350. For example, the shape of the opening 350 can correspond to a shape of a cross-section of the open channel 240, for example, a c-shaped or u-shaped opening, and/or other shapes that the open channel 240 can fit within, such as a circular opening, a square or rectangular opening (e.g., for u-shaped sleeve shafts 220), etc. In this way, the second portion 344 of the seal block 304 can surround (e.g., circumscribe) the outer surface of the sleeve shaft 220.

In some examples, the wedge 358 can extend axially along the entire length of the seal block 304. For example, the first portion 340 and the second portion 344 of the seal block 304 can include the wedge 358. In some examples, as shown in FIG. 20, the axial length of the first portion 340 of the seal block 304 is equal to an axial length of the slot 336 of the sealing member 302, such that the second end 312 of the sealing member 302 can contact an intermediate surface 360 of the seal block 304. The intermediate surface 360 can define the transition between the first portion 340 and the second portion 344 of the seal block 304. In the illustrated example, and in examples where the axial length of the first portion 340 is longer than an axial length of the slot 336 of the sealing member 302, the inner projection 330 (and the engaging surface 334 thereof) does not extend into the opening 350. In other examples, the axial length of the first portion 340 can be shorter than the axial length of the slot 336, such that the engaging surface 334 of the inner projection 330 extends at least partially into the opening 350.

The second portion 344 of the seal block 304 can have a cylindrical outer surface. In some examples, as shown in FIG. 16, an outer diameter of the second portion 344 can be less than the inner diameter of the inner wall 256 of the seal housing 252 to provide a clearance or gap for assembly and/or manufacturing purposes. In other examples, the outer surface of the second portion 344 can contact the inner wall 256 of the seal housing 252.

When the sealing member 302 and the seal block 304 are coupled together, the wedge 358 contacts the inner surface that defines the slot 336. Specifically, the wedge 358 can be configured to hold the inner projection 330 in place relative to the seal housing 252 to ensure a seal between the engaging surface 334 of the inner projection 330 and the inwardly-facing surface 244 of the open channel 240. In some examples, the shape of the slot 336 can correspond to the shape of the wedge 358. In other examples, the slot 336 can be tapered or stepped, such that the slot 336 is wider at a location closer to the outer surface of the sealing member 302 and is narrower towards the lower end of the slot 336. In these examples, the wedge 358 can stretch or expand the narrow portion of the slot 336 radially outwards against the inwardly-facing surface 244 of the open channel 240.

The sealing member 302 and the seal block 304 can be coupled together, for example, such that the wedge 358 is positioned within the slot 336, the second end 312 of the sealing member 302 contacts the intermediate surface 360, and/or the seal block 304 contacts the second intermediate surface 338. In some examples, the sealing member 302 and the seal block 304 can be coupled together after the sleeve shaft 220 is slidably inserted through the opening 316 of the sealing member 302 and the opening 350 of the seal block 304. For example, after being coupled to the sleeve shaft 220, the sealing member 302 and the seal block 304 can be translated axially until the wedge 356 of the seal block 304 is axially overlapped with the inner projection 330 of the sealing member 302, the second end 312 of the sealing member 302 contacts the intermediate surface 360, and/or the seal block 304 contacts the second intermediate surface 338.

In some instances, the clearance between the seal block 304 and the sleeve shaft 220 can be relatively small such that it may be difficult to axially overlap the wedge 358 over the relatively soft inner projection 330 if the sleeve shaft 220 is extended through the openings 316, 350 prior to coupling the sealing member 302 and the seal block 304. In some examples, to facilitate an easier coupling of the sealing member 302 and the seal block 304 together, the sealing member 302 and the seal block 304 can be coupled together prior to being coupled to the open channel 240 in some instances. For example, after the wedge 358 of the seal block 304 is axially overlapped with the inner projection 330 of the sealing member 302, the sleeve shaft 220 can be slidably inserted through the opening 316 of the sealing member 302 and the opening 350 of the seal block 304. When the sleeve shaft 220 is positioned within the openings 316, 350, the engaging surface 334 of the inner projection 330 can contact the inwardly-facing surface 244.

As described above, the wedge 358 of the seal block 304 can function similarly to the wedge 258 of the upper segment 252a of the seal housing 252 (FIG. 15) to compress a portion of a sealing member against the inwardly-facing surface 244 of the open channel 240. Due to the similar functionality of the wedge 258 of the seal housing 252 and the wedge 358 of the seal block 304, in some examples, the seal block 304 can be used in lieu of the wedge 258. For example, either wedge can be used to compress an engaging surface of a sealing member (e.g., engaging surface 134, engaging surface 334, etc.) against the inwardly-facing surface 244 of the open channel 240. Specifically, while hemostatic seal 100 was described above with reference to the wedge 258, the hemostatic seal 100 can be used with the wedge 358 of the seal block 304, in some examples. For example, the seal block 304 can be coupled to the second end 124 of the internal sealing member 104, such that the wedge 358 is positioned within the slot 136. As another example, the sealing member 302 can be positioned within the chamber defined by the upper segment 252a and the lower segment 252b, such that the wedge 258 extends within the slot 336. In this example, the seal block 304 can be omitted. In some examples, both the wedge 258 and the wedge 358 can be positioned within a slot (e.g., slot 136, slot 336) of a sealing member. For example, the wedge 258 of the seal housing 252 can be positioned axially between the sealing member (e.g., sealing member 302, internal sealing member 104) and the wedge 358 of the seal block 304 within the slot of the sealing member.

In some examples, a seal can be created between an engaging surface of an inner projection of a seal and the inwardly-facing surface 244 of the sleeve shaft 220 that is sufficient to provide hemostasis, without requiring a more rigid component or wedge being positioned within the inner projection. For example, the chamber of the seal housing 252 can apply a sufficient, constant force to the seal, such that an inner projection of the seal applies a sufficient sealing force to the inwardly-facing surface 244 during relative axial movement between the sleeve shaft 220 and the seal (e.g., a passive seal).

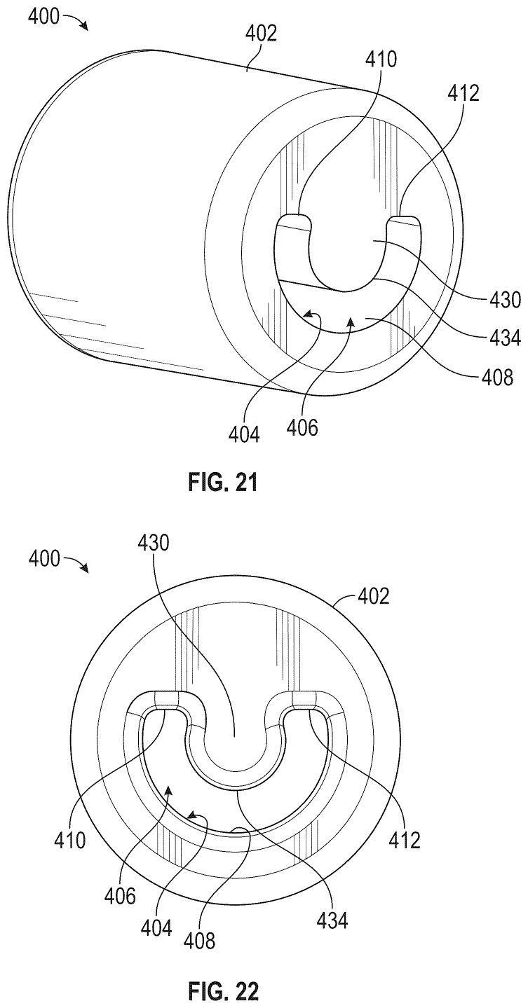

FIGS. 21-22 illustrate an example of a passive hemostatic seal 400 that can be positioned within the seal housing 252. The hemostatic seal 400 includes an outer surface 402 and an inner surface 404 defining an opening 406. The outer surface 402 can contact and/or seal against the inner wall 256 of the seal housing 252 when disposed therein. The open channel 240 of the sleeve shaft 220 can extend through the opening 406.

The seal 400 can include an inner projection 430 configured to seal against the inwardly-facing surface 244 of the open channel 240. For example, the inner projection 330 can extend radially inwards from an outer surface of the seal 400, for example, in a direction perpendicular to a longitudinal axis of the seal 400 (and perpendicular to a longitudinal axis 225 of the sleeve shaft 220). In some examples, as depicted, the inner projection 430 extends axially along the entire length of the seal 400, from a first end to a second end of the seal 400. In other examples, the inner projection 430 can extend a portion of the entire length of the seal 400. When the hemostatic seal 400 is coupled to the sleeve shaft 220, the inner projection 430 extends axially along a length of the open channel 240. Specifically, when the sleeve shaft 220 is disposed within the opening 406 of the seal 400, a first engaging portion 434 of the inner surface 404 can contact the inwardly-facing surface 244 of the open channel 240. As shown, the engaging surface 434 can generally correspond to and complement the shape of the inwardly-facing surface 244. For example, the engaging surface 434 can have an outwardly-facing surface (e.g., convex curvature, etc.). In this way, the inner projection 430 can extend or project into the opening defined by the open channel 240 and the first engaging surface 434 can seal against the inwardly-facing surface 244. In some instances, the inner projection 430, or a portion thereof, can comprise a different material (e.g., stiffer, more rigid, etc.) than the rest of the seal 400.

The shape of the opening 406 can correspond to the shape of a cross-section of the open channel 240, as a U or C-shape or incomplete (e.g., partial) annulus. For example, the inner surface 404 can contact and/or seal against all sides of the open channel 240 (e.g., surfaces 244, 246 and edges 248). For example, the first engaging portion 434 can seal against the inwardly-facing surface 244, a second engaging portion 410 (e.g., a concave, curved portion, etc.) can seal against the outwardly-facing surface 246, and third engaging portions 412 disposed on either side of the first engaging portion 434 can seal against the edges 248.

When the seal 400 is disposed within the seal housing 252, the inner wall 256, distal surface 262 and/or proximal surface 264 can apply a substantially constant force to the seal 400 (e.g., rather than a selective, compressive locking force as applied by the locking knob 254) to the seal 400 to provide a passive hemostatic seal around the open channel 240 of the sleeve shaft 220. In this way, hemostasis can be maintained as the sleeve shaft 220 moves relative to the seal 400 and the seal housing 252.

As described above, the sleeve shaft 220 can be translated axially relative to other components of the delivery apparatus 200 (e.g., relative to the pusher shaft 212, etc.) during various operations of the delivery apparatus 200. For example, during implantation of a docking device (e.g., docking device 52), a user may need to operate the sleeve shaft 220 by moving the sleeve shaft 220 in the axial direction relative to other components of the delivery apparatus 200 multiple times and/or with a high degree of fidelity or control. In some examples, the seals described herein (e.g., seal 100, seal 2400, seal 300, seal 400, etc.) exert a relatively high force on the sleeve shaft 220 to hemostatically seal around the open channel 240 of the sleeve shaft 220. This sealing force can increase the force required to axially move the sleeve shaft 220 relative to other components of the delivery apparatus 200.

To reduce the force needed to translate the sleeve shaft 220 relative to other components of the delivery apparatus 200 while maintaining the hemostatic seal around the sleeve shaft 220, a portion of the sleeve shaft 220 can include a coating 270 to improve the lubriciousness of the sleeve shaft 220 in some examples. FIGS. 23 and 24 illustrate the sleeve shaft 220 in greater detail. The lubricious coating 270 can be disposed on the outer surface of the sleeve shaft 220 along a length of the sleeve shaft 220, such as along a portion of the sleeve shaft 220 that extends through the seal (e.g., the open channel 240). Specifically, the coating 270 can be located on the open channel 240 of the sleeve shaft 220. In some examples, the coating 270 can extend proximally from the transition between the distal segment 250 of the sleeve shaft 220 and the open channel 240 towards a proximal end of the sleeve shaft 220.

In some examples, the coating 270 can extend proximally towards the sleeve handle 224, but not fully to the proximal end of the sleeve shaft 220. In this way, the location where the sleeve handle 224 is coupled to the sleeve shaft 220 (e.g., at the proximal end of the sleeve shaft 220) does not include the lubricious coating 270, to ensure a sufficient connection between the sleeve handle 224 and the sleeve shaft 220. In some examples, the coating 270 can extend to the proximal end of the sleeve shaft 220. In some examples, the coating 270 can also be disposed on a portion of the distal segment 250.

The coating 270 can be a material that maintains the lubricity of the sleeve shaft 220 during operation of the delivery apparatus 200, such that the lubricity is maintained for multiple axial movements of the sleeve shaft 220. In some examples, the coating 270 can be a hydrophilic material. In some examples, the coating 270 can be a non-stick material. In some examples, the coating 270 can be a polytetrafluoroethylene (PTFE) coating or other material that improves the lubriciousness of the sleeve shaft 220.

FIG. 24 illustrates a cross-sectional view of the sleeve shaft 220 taken through the open channel 240. As shown, the coating 270 surrounds or encapsulates the outer surfaces of the open channel 240, such that the coating 270 is radially outward of the sleeve shaft 220. Specifically, the coating 270 is located on the inwardly-facing surface 244, the outwardly-facing surface 246, and the edges 248 of the open channel 240. In this way, the coating 270 defines an outer surface that is radially outwards of the outer surfaces of the sleeve shaft 220. Thus, when a seal (e.g., any of the seals described herein) is coupled to the open channel 240 of the sleeve shaft 220, the coating 270 is positioned between the sleeve shaft 220 and other components of the delivery apparatus 200, to improve the lubricity of the sleeve shaft 220. In some examples, the coating 270 can be considered the outer surface of the sleeve shaft 220.

In some examples, the delivery apparatus 200 can include a lubricant to improve the lubricity of the sleeve shaft 220. For example, as shown in FIG. 25, the delivery apparatus 200 can include a lubricant chamber 272 positioned adjacent to the hemostatic seal 500 that includes a supply of lubricant 274. The lubricant 274 can be a PTFE grease, silicone oil, or the like. In the illustrated example, the lubricant chamber 272 is positioned within the seal housing 252 and is located axially between two sealing members of the hemostatic seal 500. One or both of the sealing members of the hemostatic seal 500 can be any of the hemostatic seals described herein (e.g., seal 100, seal 2400, seal 300, seal 400, etc.). As one example, the sealing member of the hemostatic seal 500 that is positioned distal to the lubricant chamber 272 can be configured as seal 400 and the sealing member of the hemostatic seal 500 that is positioned proximal to the lubricant chamber 272 can be configured as seal 100. In some examples, the lubricant 274 can be used in addition to or in lieu of the coating 270.

The lubricant chamber 272 can be defined by an inner surface of the seal housing 252. In some examples, as shown in FIG. 25, the lubricant chamber 272 can help retain the axial position of the seal 500 relative to the seal housing 252. For example, the lubricant chamber 272 can have a diameter than is smaller than an outer diameter of the seal 500, such that the seal 500 can be positioned against the inner surface of the seal housing 252 that defines the lubricant chamber 272. In this way, the lubricant chamber 272 can retain a volume of lubricant 274 and can help prevent the seal 500 from moving relative to the seal housing 252 (e.g., during movement of the sleeve shaft 220). In some examples, the seal housing 252 can maintain the positioning of the seal 500 relative to the seal housing 252 using other structures (e.g., walls, flanges, lips, etc.) independent of the lubricant chamber 272, for example, as described above. In some examples, the lubricant chamber 272 can have a diameter that is equal to or greater than the outer diameter of the seal 500.

The open channel 240 of the sleeve shaft 220 is in fluid communication with the lubricant chamber 272. In this way, as the sleeve shaft 220 moves axially relative to the seal housing 252 (and therefore the lubricant chamber 272), the lubricant 274 within the lubricant chamber 272 can coat the open channel 240 of the sleeve shaft 220 and improve the lubricity thereof. In this way, the lubricant 274 can be applied to the outer surface of the open channel 240.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. In some examples, the sealing members described herein can be made from silicone, which enables diffusion of ethylene oxide through the material and into the medical device. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus comprising: a seal housing; a first shaft extending through the seal housing and comprising an outwardly-facing surface and an inwardly-facing surface, wherein the inwardly-facing surface defines an open channel; a second shaft comprising a first segment and a second segment, wherein the first segment is disposed within the open channel, and wherein the second segment extends out of the open channel and is angled relative to the first segment; and a seal coupled to the first shaft, the seal including a first sealing portion and a second sealing portion, wherein the first sealing portion seals a first gap between the seal housing and the outwardly-facing surface of the first shaft, wherein the second sealing portion seals a second gap between the seal housing and the inwardly-facing surface of the first shaft, wherein the seal provides homeostasis when the first shaft moves relative to the seal.

Example 2. The delivery apparatus of any example herein, particularly example 1, wherein the seal is compressed against the first shaft when the first shaft moves relative to the seal.

Example 3. The delivery apparatus of any example herein, particularly either example 1 or example 2, wherein the outwardly-facing surface of the first shaft is convex, and wherein the inwardly-facing surface of the first shaft is concave.

Example 4. The delivery apparatus of any example herein, particularly any one of examples 1-3, wherein the second sealing portion comprises an inner projection, wherein the inner projection includes a slot and an engaging surface, wherein the engaging surface of the inner projection contacts the inwardly-facing surface of the first shaft.

Example 5. The delivery apparatus of any example herein, particularly example 4, wherein a portion of the seal housing extends radially into the slot, wherein the portion of the seal housing is configured to compress the engaging surface against the inwardly-facing surface of the first shaft.

Example 6. The delivery apparatus of any example herein, particularly either example 4 or example 5, further comprising a seal block coupled to the seal, wherein the seal block includes a wedge, and wherein the wedge is positioned within the slot.

Example 7. The delivery apparatus of any example herein, particularly example 6, wherein a durometer hardness of the seal is lower than a durometer hardness of the seal block.

Example 8. The delivery apparatus of any example herein, particularly any one of examples 1-7, further comprising a locking mechanism operatively coupled to the first shaft to prevent movement of the first shaft relative to the seal, wherein the seal provides homeostasis independent of the locking mechanism.

Example 9. A delivery apparatus comprising: a seal housing; a first shaft extending through the seal housing and comprising an outwardly-facing surface and an inwardly-facing surface, wherein the inwardly-facing surface defines an open channel; a second shaft comprising a first segment and a second segment, wherein the first segment is disposed within the open channel, and wherein the second segment extends out of the open channel and is angled relative to the first segment; and a seal assembly coupled to the first shaft, the seal including a first sealing member and a second sealing member, wherein the first sealing member seals a first gap between the seal housing and the outwardly-facing surface of the first shaft, wherein the second sealing member seals a second gap between the seal housing and the inwardly-facing surface of the first shaft, wherein the seal provides homeostasis when the first shaft moves relative to the seal.

Example 10. The delivery apparatus of any example herein, particularly example 9, wherein the seal housing compresses the seal assembly against the first shaft when the first shaft moves relative to the seal.

Example 11. The delivery apparatus of any example herein, particularly either example 9 or example 10, wherein a cross-section of the first shaft is partially annular.

Example 12. The delivery apparatus of any example herein, particularly any one of example 9-11, wherein the outwardly-facing surface of the first shaft is convex, and wherein the inwardly-facing surface of the first shaft is concave.

Example 13. The delivery apparatus of any example herein, particularly any one of examples 9-12, wherein the second sealing member comprises an inner projection having an engaging surface, wherein the engaging surface contacts the inwardly-facing surface of the first shaft.

Example 14. The delivery apparatus of any example herein, particularly example 13, wherein the second sealing member comprises an opening extending axially from a first end of the second sealing member to a second end of the second sealing member, wherein the first shaft extends through the opening of the second sealing member.

Example 15. The delivery apparatus of any example herein, particularly example 14, wherein the inner projection extends radially inwards into the opening, and wherein the inner projection is disposed at the second end of the second sealing member.

Example 16. The delivery apparatus of any example herein, particularly example 15, wherein the second sealing member comprises an intermediate surface disposed axially between the first end and the second end, wherein an axial end of the inner projection defines the intermediate surface.

Example 17. The delivery apparatus of any example herein, particularly example 16, wherein the second sealing member comprises a slot extending axially along a length of the inner projection.

Example 18. The delivery apparatus of any example herein, particularly example 17, wherein the slot is an axial through-slot that extends through an entire length of the inner projection, such that a first end of the slot is defined by the intermediate surface and a second end of the slot is defined by the second end of the second sealing member.

Example 19. The delivery apparatus of any example herein, particularly example 17, wherein an axial length of the slot is less than an entire length of the inner projection.

Example 20. The delivery apparatus of any example herein, particularly any one of examples 17-19, wherein the seal housing comprises an inner wall defining a chamber, wherein the seal assembly is positioned within the chamber, and wherein the seal assembly contacts the inner wall.

Example 21. The delivery apparatus of any example herein, particularly example 20, wherein the inner wall includes a wedge, wherein the wedge projects radially inwards into the slot, wherein the wedge of the seal housing is configured to compress the engaging surface against the inwardly-facing surface of the first shaft.

Example 22. The delivery apparatus of any example herein, particularly any one of examples 17-21, further comprising a seal block coupled to the seal assembly, wherein the seal block includes a wedge, and wherein the wedge of the seal block is positioned within the slot.

Example 23. The delivery apparatus of any example herein, particularly example 22, wherein a durometer hardness of the seal assembly is lower than a durometer hardness of the seal block.

Example 24. The delivery apparatus of any example herein, particularly any one of examples 9-23, wherein the first sealing member and the second sealing member are partially overlapped in the axial direction.

Example 25. The delivery apparatus of any example herein, particularly any one of examples 9-23, wherein the first sealing member comprises an inner surface defining an opening, wherein the first shaft extends through the opening of the first sealing member.

Example 26. The delivery apparatus of any example herein, particularly example 25, wherein the opening of the first sealing member is D-shaped.

Example 27. The delivery apparatus of any example herein, particularly either example 25 or example 26, wherein the inner surface of the first sealing member comprises a step.

Example 28. The delivery apparatus of any example herein, particularly example 27, wherein an end of the second sealing member contacts the step.

Example 29. The delivery apparatus of any example herein, particularly any one of examples 9-28, further comprising a handle, wherein the handle comprises a straight segment and a branched segment that is angled relative to the straight segment, wherein the first shaft extends through the straight segment, wherein the second shaft is at least partially disposed within the branched segment.

Example 30. The delivery apparatus of any example herein, particularly example 29, wherein the seal housing is coupled to the straight segment.

Example 31. The delivery apparatus of any example herein, particularly any one of examples 9-29, further comprising a locking mechanism operatively coupled to the first shaft to prevent movement of the first shaft relative to the seal, wherein the seal provides homeostasis independent of the locking mechanism.

Example 32. A delivery apparatus comprising: a seal housing; a shaft extending through the seal housing, wherein the shaft comprises an outer surface, wherein the outer surface comprises an inwardly-facing portion and an outwardly-facing portion; and a sealing member disposed within the seal housing, wherein the sealing member includes an inner surface defining an opening, wherein the shaft extends through the opening of the sealing member, wherein the sealing member includes an inner projection having an engaging surface, wherein the engaging surface seals against the inwardly-facing portion of the outer surface of the shaft, wherein the sealing member provides hemostasis when the shaft moves relative to the sealing member.

Example 33. The delivery apparatus of any example herein, particularly example 32, further comprising an external sealing member coupled to the sealing member, wherein the external sealing member includes an inner surface defining an opening, wherein the shaft extends through the opening of the external sealing member, wherein the inner surface seals against the outwardly-facing portion of the outer surface of the shaft.

Example 34. The delivery apparatus of any example herein, particularly example 33, wherein the opening of the external sealing member is D-shaped.

Example 35. The delivery apparatus of any example herein, particularly any one of examples 32-34, wherein the inner projection includes a slot extending in an axial direction along a length of the inner projection.

Example 36. The delivery apparatus of any example herein, particularly example 35, wherein the length is less than an entire length of the inner projection.

Example 37. The delivery apparatus of any example herein, particularly example 35, wherein the slot extends an entire length of the inner projection.

Example 38. The delivery apparatus of any example herein, particularly any one of examples 35-37, further comprising a seal block coupled to the sealing member, wherein the seal block includes a wedge, wherein the wedge is positioned within the slot.

Example 39. The delivery apparatus of any example herein, particularly example 38, wherein the seal block has a higher durometer hardness than the sealing member.

Example 40. The delivery apparatus of any example herein, particularly either example 38 or example 39, wherein the engaging surface is positioned radially between the inwardly-facing portion of the shaft and the wedge.

Example 41. A seal assembly for a delivery apparatus, the seal assembly comprising: a first sealing member, the first sealing member defining a first opening extending through the first sealing member in an axial direction, wherein the first opening comprises an inwardly-facing surface, the inwardly-facing surface configured to seal against an outwardly-facing surface of a shaft; and a second sealing member coupled to the first sealing member, the second sealing member defining a second opening extending through the second sealing member in the axial direction, wherein the second sealing member comprises an inner projection extending into the second opening in a radial direction, wherein the inner projection includes an outwardly-facing, engaging surface, wherein the engaging surface is configured to seal against an inwardly-facing surface of the shaft.

Example 42. The seal assembly of any example herein, particularly example 41, wherein the second sealing member has a first end, a second end, and an intermediate surface disposed axially between the first end and the second end, and wherein the inner projection is disposed at the second end of the second sealing member and defines the intermediate surface.

Example 43. The seal assembly of any example herein, particularly example 42, wherein the inner projection includes a slot extending along an axial length of the inner projection, wherein the slot is open in a radial direction through an outer surface of the second sealing member.

Example 44. The seal assembly of any example herein, particularly example 43, wherein the slot defines a third opening extending through the second sealing member in the axial direction.

Example 45. The seal assembly of any example herein, particularly any one of examples 41-44, wherein the second sealing member comprises an axial extension at the first end of the second sealing member, the axial extension disposed around an outer surface of the first sealing member.

Example 46. The seal assembly of any example herein, particularly example 45, wherein the outer surface of the first sealing member comprises a step.

Example 47. The seal assembly of any example herein, particularly example 46, wherein the axial extension contacts the step when the first and second sealing members are coupled together.

Example 48. The seal assembly of any example herein, particularly any one of examples 40-47, wherein the first opening is D-shaped.

Example 49. The seal assembly of any example herein, particularly any one of examples 40-48, wherein the second opening is partially annular.

Example 50. A seal for a delivery apparatus, the seal comprising: a body, the body having a first sealing portion and a second sealing portion, the first sealing portion axially spaced apart from the second sealing portion, wherein the first sealing portion comprises an opening having inwardly-facing surface, and wherein the second sealing portion comprises an inner projection having an outwardly-facing surface.

Example 51. The seal of any example herein, particularly example 50, wherein the opening is D-shaped.

Example 52. The seal of any example herein, particularly either example 50 or 51, wherein the inner projection includes a slot extending along an axial length of the inner projection, wherein the slot is open in a radial direction through an outer surface of the seal.

Example 53. The seal of any example herein, particularly example 52, wherein the slot is open in an axial direction through an end of the seal.

Example 54. The delivery apparatus of any example herein, particularly any one of examples 1-40, wherein the delivery apparatus is sterilized.

Example 55. A delivery apparatus comprising: a sleeve shaft comprising a first segment and a second segment, wherein the second segment comprises an inwardly-facing outer surface and an outwardly-facing outer surface, wherein the second segment comprises a lubricious coating; and a seal coupled to the second segment of the sleeve shaft.

Example 56. The delivery apparatus of any example herein, particularly example 55, wherein the first segment has an annular cross-section, and wherein the second segment has a partially annular cross-section.

Example 57. The delivery apparatus of any example herein, particularly either example 55 or example 56, wherein the inwardly-facing outer surface is concave and the outwardly-facing outer surface is convex.

Example 58. The delivery apparatus of any example herein, particularly any one of examples 55-57, wherein the lubricious coating comprises a PTFE coating.

Example 59. The delivery apparatus of any example herein, particularly any one of examples 55-58, wherein the sleeve shaft is axially moveable relative to the seal.

Example 60. The delivery apparatus of any example herein, particularly any one of examples 55-59, further comprising a seal housing, wherein the sleeve shaft extends through the seal housing, and wherein the seal is disposed within the seal housing.

Example 61. The delivery apparatus of any example herein, particularly example 60, wherein the seal includes a first sealing portion and a second sealing portion, wherein the first sealing portion seals a first gap between the seal housing and the outwardly-facing outer surface of the sleeve shaft, wherein the second sealing portion seals a second gap between the seal housing and the inwardly-facing outer surface of the sleeve shaft, wherein the seal provides homeostasis when the sleeve shaft moves relative to the seal.

Example 62. The delivery apparatus of any example herein, particularly example 60, wherein the seal includes an inner surface defining an opening, wherein the sleeve shaft extends through the opening of the seal, wherein the seal includes an inner projection having an engaging surface, wherein the engaging surface seals against the inwardly-facing outer surface of the sleeve shaft, and wherein the seal provides hemostasis when the sleeve shaft moves relative to the seal.

Example 63. The delivery apparatus of any example herein, particularly example 60, wherein the seal comprises a first sealing member and a second sealing member, wherein the first sealing member seals a first gap between the seal housing and the outwardly-facing outer surface of the sleeve shaft, wherein the second sealing member seals a second gap between the seal housing and the inwardly-facing outer surface of the sleeve shaft, wherein the seal provides homeostasis when the sleeve shaft moves relative to the seal.

Example 64. The delivery apparatus of any example herein, particularly any one of examples 55-63, further comprising a second shaft that is axially moveable relative to the sleeve shaft and extends through the first segment of the sleeve shaft, wherein a portion of the second shaft is angled relative to the sleeve shaft.

Example 65. A delivery apparatus comprising: a seal housing defining a lubricant chamber including a lubricant; a seal disposed within the seal housing; and a sleeve shaft extending through the seal housing, the sleeve shaft comprising a first segment and a second segment, wherein the second segment extends through the lubricant chamber and the seal, wherein the second segment comprises an inwardly-facing outer surface and an outwardly-facing outer surface.

Example 66. The delivery apparatus of any example herein, particularly example 65, wherein the first segment has an annular cross-section, and wherein the second segment has a partially annular cross-section.

Example 67. The delivery apparatus of any example herein, particularly either example 65 or example 66, wherein the inwardly-facing outer surface is concave and the outwardly-facing outer surface is convex.

Example 68. The delivery apparatus of any example herein, particularly any one of examples 65-67, wherein the lubricant comprises a PTFE grease or a silicone oil.

Example 69. The delivery apparatus of any example herein, particularly any one of examples 65-68, wherein the sleeve shaft is axially moveable relative to the seal.

Example 70. The delivery apparatus of any example herein, particularly any one of examples 65-69, wherein the seal includes a first sealing member positioned distal to the lubricant chamber and wherein the seal includes a second sealing member positioned proximal to the lubricant chamber.

Example 71. The delivery apparatus of any example herein, particularly any one of examples 65-70, wherein the seal includes a first sealing portion and a second sealing portion, wherein the first sealing portion seals a first gap between the seal housing and the outwardly-facing outer surface of the sleeve shaft, wherein the second sealing portion seals a second gap between the seal housing and the inwardly-facing outer surface of the sleeve shaft, wherein the seal provides homeostasis when the sleeve shaft moves relative to the seal.

Example 72. The delivery apparatus of any example herein, particularly any one of examples 65-70, wherein the seal includes an inner surface defining an opening, wherein the sleeve shaft extends through the opening of the seal, wherein the seal includes an inner projection having an engaging surface, wherein the engaging surface seals against the inwardly-facing outer surface of the sleeve shaft, and wherein the seal provides hemostasis when the sleeve shaft moves relative to the seal.

Example 73. The delivery apparatus of any example herein, particularly example 70, wherein the first sealing member seals a first gap between the seal housing and the outwardly-facing outer surface of the sleeve shaft, wherein the second sealing member seals a second gap between the seal housing and the inwardly-facing outer surface of the sleeve shaft, wherein the seal provides homeostasis when the sleeve shaft moves relative to the seal.

Example 74. The delivery apparatus of any example herein, particularly any one of examples 65-73, further comprising a second shaft that is axially moveable relative to the sleeve shaft and extends through the first segment of the sleeve shaft, wherein a portion of the second shaft is angled relative to the sleeve shaft.

Example 75. The delivery apparatus of any example herein, particularly any one of examples 55-74, wherein the delivery apparatus is sterilized.

The features described herein with regard to any example can be combined with other features described in any one or more of the other examples, unless otherwise stated. For example, any one or more of the features of one shaft can be combined with any one or more features of another shaft. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A delivery apparatus comprising:
a seal housing;
a first shaft extending through the seal housing and comprising an outwardly-facing surface and an inwardly-facing surface, wherein the inwardly-facing surface defines an open channel;
a second shaft comprising a first segment and a second segment, wherein the first segment is disposed within the open channel, and wherein the second segment extends out of the open channel and is angled relative to the first segment; and
a seal coupled to the first shaft, the seal including a first sealing portion and a second sealing portion, wherein the first sealing portion seals a first gap between the seal housing and the outwardly-facing surface of the first shaft, wherein the second sealing portion seals a second gap between the seal housing and the inwardly-facing surface of the first shaft, wherein the seal provides homeostasis when the first shaft moves relative to the seal.

2. The delivery apparatus of claim 1, wherein the seal is compressed against the first shaft when the first shaft moves relative to the seal.

3. The delivery apparatus of claim 1, wherein the outwardly-facing surface of the first shaft is convex, and wherein the inwardly-facing surface of the first shaft is concave.

4. The delivery apparatus of claim 1, wherein the second sealing portion comprises an inner projection, wherein the inner projection includes a slot and an engaging surface, wherein the engaging surface of the inner projection contacts the inwardly-facing surface of the first shaft.

5. The delivery apparatus of claim 4, wherein a portion of the seal housing extends radially into the slot, wherein the portion of the seal housing is configured to compress the engaging surface against the inwardly-facing surface of the first shaft.

6. The delivery apparatus of claim 4, further comprising a seal block coupled to the seal, wherein the seal block includes a wedge, and wherein the wedge is positioned within the slot.

7. The delivery apparatus of claim 6, wherein a durometer hardness of the seal is lower than a durometer hardness of the seal block.

8. The delivery apparatus of claim 1, further comprising a locking mechanism operatively coupled to the first shaft to prevent movement of the first shaft relative to the seal, wherein the seal provides homeostasis independent of the locking mechanism.

9. A delivery apparatus comprising:
a seal housing;
a shaft extending through the seal housing, wherein the shaft comprises an outer surface, wherein the outer surface comprises an inwardly-facing portion and an outwardly-facing portion; and
a sealing member disposed within the seal housing, wherein the sealing member includes an inner surface defining an opening, wherein the shaft extends through the opening of the sealing member, wherein the sealing member includes an inner projection having an engaging surface, wherein the engaging surface seals against the inwardly-facing portion of the outer surface of the shaft, wherein the sealing member provides hemostasis when the shaft moves relative to the sealing member.

10. The delivery apparatus of claim 9, further comprising an external sealing member coupled to the sealing member, wherein the external sealing member includes an inner surface defining an opening, wherein the shaft extends through the opening of the external sealing member, wherein the inner surface seals against the outwardly-facing portion of the outer surface of the shaft.

11. The delivery apparatus of claim 10, wherein the opening of the external sealing member is D-shaped.

12. The delivery apparatus of claim 9, wherein the inner projection includes a slot extending in an axial direction along a length of the inner projection.

13. The delivery apparatus of claim 12, wherein the length is less than an entire length of the inner projection.

14. The delivery apparatus of claim 12, wherein the slot extends an entire length of the inner projection.

15. The delivery apparatus of claim 12, further comprising a seal block coupled to the sealing member, wherein the seal block includes a wedge, wherein the wedge is positioned within the slot.

16. The delivery apparatus of claim 15, wherein the seal block has a higher durometer hardness than the sealing member.

17. The delivery apparatus of claim 15, wherein the engaging surface is positioned radially between the inwardly-facing portion of the shaft and the wedge.

18. A seal assembly for a delivery apparatus, the seal assembly comprising:
a first sealing member, the first sealing member defining a first opening extending through the first sealing member in an axial direction, wherein the first opening comprises an inwardly-facing surface, the inwardly-facing surface configured to seal against an outwardly-facing surface of a shaft; and
a second sealing member coupled to the first sealing member, the second sealing member defining a second opening extending through the second sealing member in the axial direction, wherein the second sealing member comprises an inner projection extending into the second opening in a radial direction, wherein the inner projection includes an outwardly-facing, engaging surface, wherein the engaging surface is configured to seal against an inwardly-facing surface of the shaft.

19. The seal assembly of claim 18, wherein the second sealing member has a first end, a second end, and an intermediate surface disposed axially between the first end and the second end, and wherein the inner projection is disposed at the second end of the second sealing member and defines the intermediate surface.

20. A seal for a delivery apparatus, the seal comprising:
a body, the body having a first sealing portion and a second sealing portion, the first sealing portion axially spaced apart from the second sealing portion, wherein the first sealing portion comprises an opening having inwardly-facing surface, and wherein the second sealing portion comprises an inner projection having an outwardly-facing surface.
